(19) United States
(12) Patent Application Publication
(10) Pub. No.: US 2019/0104748 A1
(43) Pub. Date: Apr. 11, 2019

(54) Methods of Controlling the Rate of Ripening in Harvested Produce

(71) Applicant: Apeel Technology, Inc., Goleta, CA (US)

(72) Inventors: Stephen William Kaun, Santa Barbara, CA (US); Chance Holland, Goleta, CA (US); James T. Rogers, Santa Barbara, CA (US); Louis Perez, Santa Barbara, CA (US); Carlos Hernandez, Santa Barbara, CA (US); Charles Frazier, Goleta, CA (US)

(21) Appl. No.: 16/209,794

(22) Filed: Dec. 4, 2018

Related U.S. Application Data
(63) Continuation of application No. PCT/US2017/041167, filed on Jul. 7, 2017.
(60) Provisional application No. 62/359,898, filed on Jul. 8, 2016.

(51) Int. Cl. A23B 7/16; A23B 7/154; C07C 69/28; C07D 303/16; C07C 69/675; C07C 69/533

(52) U.S. Cl. CPC A23B 7/16; A23B 7/154; C07C 69/28; A23V 2002/00; C07C 69/675; C07C 69/533; C07D 303/16

(57) ABSTRACT

The present disclosure provides methods for controlling the rate of ripening for agricultural produce. The present disclosure further provides coating compositions that can be applied to produce to control (e.g., lessen) the rate of ripening of the produce.

### CROSS REFERENCE TO RELATED APPLICATIONS

This application claims priority to, and the benefit of, PCT Application No. PCT/US2017/041167, filed Jul. 7, 2017, which claims priority to U.S. Provisional Application No. 62/359,898, filed Jul. 8, 2016, the contents of which are hereby incorporated by reference in their entirety.

### FIELD OF THE DISCLOSURE

The present disclosure relates to formulations and methods for treating agricultural products, such as produce, in order to control the rate of ripening.

### BACKGROUND

Many common agricultural products, for example avocados and bananas, are typically harvested prior to complete ripening and then allowed to fully ripen post-harvest, for example during storage or shipping. Since many of these products are seasonal, and hence only ripen during a limited time window, it may be desirable to delay the ripening of the products in order to make them available to consumers during times that they would not otherwise be available.

### SUMMARY

Described herein are formulations and methods for delaying and controlling the rate of ripening of agricultural products such as harvested produce. The methods generally include first forming a protective coating over the products that slows respiration, thereby inhibiting ripening agents such as oxygen or ethylene from being adsorbed into the products, as well as reducing the rate of moisture loss from the products. The protective coating can be maintained over the products for a time sufficient to delay the initiation of ripening and slow the rate of ripening of the products, after which the coating can be at least partially removed or otherwise modified to allow the products to ripen more rapidly. By staggering the times at which coatings formed over a batch of harvested products are removed or modified, ripened products from the batch can be incrementally provided to consumers over extended periods of time.

As used herein, a “coating,” “molecular coating,” or “protective coating” can refer to one or more layers of molecules, (e.g., monomers, oligomers, low molecular weight polymers, or combinations thereof) disposed over a surface of an agricultural product such as a piece of produce. In some implementations, the monomers, oligomers, low molecular weight polymers, or combinations thereof that form a coating can be modified or functionalized, as described below. For example, the monomers, oligomers, low molecular weight polymers, or combinations thereof can be of the Formula I, Formula I-A, or Formula I-B as set forth below.

In one aspect, a method of controlling the rate of ripening in harvested produce includes applying a coating composition to the produce to form a coating over a surface of the produce. The coating composition can comprise a plurality of fatty acids, esters, triglycerides, diglycerides, monoglycerides, amides, amines, thiols, thioesters, carboxylic acids, ethers, aliphatic waxes, alcohols, salts, acids, bases, proteins, enzymes, monomers, oligomers, low molecular weight polymers, or combinations thereof. The method can further comprise storing the produce and at least partially removing or otherwise modifying the coating.

In another aspect, a method of controlling the rate of ripening in harvested produce includes receiving the produce, wherein the produce has a protective coating over a surface thereof. The coating can be formed from a composition of monomers, oligomers, low molecular weight polymers, or combinations thereof. The method can further comprise storing the produce and at least partially removing or otherwise modifying the coating.

In another aspect, a method of controlling the ripening of harvested produce includes causing a coating composition to be applied to a surface of the produce. The coating composition can include a coating agent dissolved in a solvent, the coating agent formulated to form a protective coating over the surface of the produce. The method can further comprise causing the coated produce to be stored for at least 1 day, and at least partially removing or modifying the coating. The applying of the coating composition to the surface of the produce can reduce the ripening rate of the produce, and the at least partial removal or modification of the coating can cause the ripening rate of the produce to increase.

In another aspect, a method of delaying ripening of harvested produce can include applying a coating composition to a surface of the produce. The coating composition can include a coating agent in a solvent. The method can further comprise allowing the coating agent to solidify and form a coating over the surface of the produce, storing the produce, and at least partially removing or otherwise modifying the coating.

In another aspect, a method of storing produce can include applying a coating composition to a surface of the produce. The coating composition can include a coating agent in a solvent. The method can further comprise allowing the solvent to at least partially evaporate, thereby causing a coating to form from the coating agent over the surface of the produce, the coating formulated to reduce a respiration rate of the produce. The method can further include storing the produce for at least 1 day, and at least partially removing or otherwise modifying the coating, thereby increasing the respiration rate of the produce.

In another aspect, a method of storing produce can include receiving produce that includes a protective coating formed thereon. The protective coating can be formed from at least one of fatty acids, esters, triglycerides, diglycerides, monoglycerides, amides, amines, thiols, thioesters, carboxylic acids, ethers, aliphatic waxes, alcohols, salts, acids, bases, proteins, enzymes, monomers, oligomers, and low molecular weight polymers. The method further can comprise storing the produce, and at least partially removing or otherwise modifying the coating.

In another aspect, a method of treating an agricultural product can include causing a coating composition to be applied to a surface of the agricultural product. The composition can include a coating agent dissolved in a solvent, and the coating agent can be formulated to form a protective coating over the surface of the agricultural product. The method can further comprise storing the agricultural product, and at least partially removing the coating.

In another aspect, a method of storing produce can include applying a coating composition to the produce to form a protective coating over a surface of the produce. The protective coating can be formulated to cause a rate of respiration of the produce to decrease. The method can further include storing the produce for a first time period, and then modifying the protective coating to cause the rate of respiration of the produce to increase. Optionally, during the first time period, the produce can be stored at a first temperature, and the modifying of the protective coating can comprise heating the produce to a second temperature greater than the first temperature. The modifying of the protective coating can alternatively comprise at least partially removing the protective coating.

In another aspect, a method of treating an agricultural product can include applying a coating composition to a surface of the agricultural product, thereby forming a protective coating over the agricultural product, and storing the agricultural product at a first ambient temperature, wherein the protective coating is formulated to cause the ripening rate of the agricultural product to decrease while the agricultural product is stored at the first ambient temperature. The method can further include causing the ambient temperature of the agricultural product to change to a second ambient temperature, thereby reducing the efficacy of the protective coating. The second ambient temperature can be greater than or less than the first ambient temperature. Optionally, the first ambient temperature can be less than 13° C., and the second ambient temperature can be greater than 20° C. Optionally the agricultural product can be maintained at a third ambient temperature less than the second ambient temperature while the coating is formed.

In another aspect, a method of treating an agricultural product can include prior to the agricultural product being ripe, applying a coating composition to a surface of the agricultural product. The coating composition can include a coating agent in a solvent, with the coating agent formulated to form a protective coating over the surface of the agricultural product. The protective coating can serve to delay ripening of the agricultural product. The method can further include storing the agricultural product.

Methods, coatings, formulations and/or produce or other agricultural products described herein can each include one or more of the following steps or features, either alone or in combination with one another. Coatings can comprise cross-linked monomers, oligomers, low molecular weight polymers, or combinations thereof. The monomers, oligomers, low molecular weight polymers, or combinations thereof can crosslink on the surface of the produce. The harvested produce can be stored for at least 1 day with the coating thereon prior to removal of the coating. The produce can be coated before the produce is harvested, and the coating can be at least partially removed after the produce is harvested. The at least partial removal of the coating can comprise rinsing the produce in a solvent, and the solvent can optionally be heated to at least 30° C., at least 40° C., or at least 50° C. The solvent can comprise water, ethanol, or combinations thereof. The solvent can be ethanol which is cooled to 13° C. or below. The produce can be coated after the produce is harvested, and the coating can be at least partially removed prior to consumption. In some embodiments, coatings of the present disclosure can broaden the climacteric respiration peak of climacteric produce. In some embodiments, coatings of the present disclosure can widen the climacteric respiration peak of climacteric produce.

The coating can be formulated to reduce a rate of respiration of the produce. The coating can be formulated to cause a rate of respiration of the produce to decrease, and the at least partially removing of the coating can cause the rate of respiration of the produce to increase. The coating can be substantially undetectable to the human eye when applied to the produce. The coating can be substantially odorless or tasteless when applied to the produce. The coating composition can be formulated such that the coating reduces water loss from the produce. The coating composition can include at least one of monomers, oligomers, and low molecular weight polymers. The coating composition can include monoacylglycerides.

The coating composition can include a compound of Formula I:

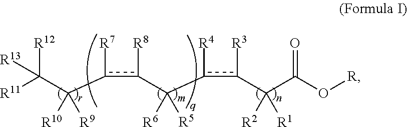

wherein:

R is selected from —H, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl or heteroaryl is optionally substituted with one or more C₁-C₆ alkyl or hydroxy;

R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;

R³, R⁴, R⁷ and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, or halogen; or

R³ and R⁴ can combine with the carbon atoms to which they are attached to form a C₃-C₆ cycloalkyl, a C₄-C₆ cycloalkenyl, or 3 to 6-membered ring, heterocycle; and/or

R⁷ and R⁸ can combine with the carbon atoms to which they are attached to form a C₃-C₆ cycloalkyl, a C₄-C₆ cycloalkenyl, or 3 to 6-membered ring, heterocycle; and/or

R¹⁴ and R¹⁵ are each independently, at each occurrence, —H, —C₁-C₆ alkyl, —C₂-C₆ alkenyl, or —C₂-C₆ alkynyl;

the symbol [symbol] represents an optionally single or cis or trans double bond;

n is 0, 1, 2, 3, 4, 5, 6, 7, or 8;

m is 0, 1, 2, or 3;

q is 0, 1, 2, 3, 4, or 5; and

r is 0, 1, 2, 3, 4, 5, 6, 7, or 8.

The coating composition can include a compound of Formula I-A:

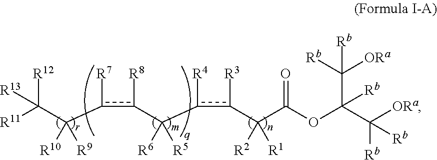

wherein:

each R^{a} is independently —H or —C₁-C₆ alkyl;

each R^{b} is independently selected from —H, —C₁-C₆ alkyl, or —OH;

R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;

R³, R⁴, R⁷ and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, —SR¹⁴, or halogen; or

R³ and R⁴ can combine with the carbon atoms to which they are attached to form a C₃-C₆ cycloalkyl, a C₄-C₆ cycloalkenyl, or 3 to 6-membered ring, heterocycle; and/or

R⁷ and R⁸ can combine with the carbon atoms to which they are attached to form a C₃-C₆ cycloalkyl, a C₄-C₆ cycloalkenyl, or 3 to 6-membered ring, heterocycle; and/or

R¹⁴ and R¹⁵ are each independently, at each occurrence, —H, —C₁-C₆ alkyl, —C₂-C₆ alkenyl, or —C₂-C₆ alkynyl;

the symbol [symbol] represents an optionally single or cis or trans double bond;

n is 0, 1, 2, 3, 4, 5, 6, 7, or 8;

m is 0, 1, 2, or 3;

q is 0, 1, 2, 3, 4, or 5; and

r is 0, 1, 2, 3, 4, 5, 6, 7, or 8.

The coating composition can include a compound of Formula I-B:

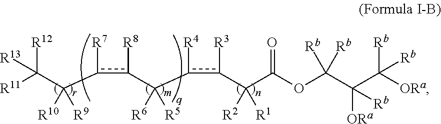

wherein:

each R^{a} is independently —H or —C₁-C₆ alkyl;

each R^{b} is independently selected from —H, —C₁-C₆ alkyl, or —OH;

R¹, R², R⁵, R⁶, R⁹, R¹⁰, R¹¹, R¹² and R¹³ are each independently, at each occurrence, −H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, or halogen;

R³, R⁴, R⁷ and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, or halogen; or

R³ and R⁴ can combine with the carbon atoms to which they are attached to form a C₃-C₆ cycloalkyl, a C₄-C₆ cycloalkenyl, or 3 to 6-membered ring, heterocycle; and/or

R⁷ and R⁸ can combine with the carbon atoms to which they are attached to form a C₃-C₆ cycloalkyl, a C₄-C₆ cycloalkenyl, or 3 to 6-membered ring, heterocycle; and/or

R¹⁴ and R¹⁵ are each independently, at each occurrence, —H, —C₁-C₆ alkyl, —C₂-C₆ alkenyl, or —C₂-C₆ alkynyl;

the symbol [symbol] represents an optionally single or cis or trans double bond;

n is 0, 1, 2, 3, 4, 5, 6, 7, or 8;

m is 0, 1, 2, or 3;

q is 0, 1, 2, 3, 4, or 5; and

r is 0, 1, 2, 3, 4, 5, 6, 7, or 8.

The protective coatings can have a thickness less than about 10 microns. The protective coating can have an average transmittance of at least 60% for light in the visible range. The methods can further comprise treating the produce with a ripening agent. The treating of the produce with the ripening agent can comprise gassing the produce with ethylene. The monomers, oligomers, low molecular weight polymers, or combinations thereof can be functionalized (e.g., by esterification, for instance with a glycerol molecule).

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a qualitative plot indicating the relative state of ripening of produce as a function of time for uncoated produce and for produce having a coating applied once the produce has ripened.

FIG. 2 is a qualitative plot indicating the relative state of ripening of produce as a function of time for uncoated produce and for produce having a coating applied prior to ripening of the produce.

FIG. 3 shows a flow chart diagramming a process for controlling or delaying ripening in produce by forming a coating over the produce and subsequently removing the coating.

FIG. 4 is a plot of mass loss rates for avocados that were coated with a formulation as described herein and then subsequently had their coatings removed by soaking in water.

FIG. 5 is a plot of the percent area reduction as a function of time for the avocados measured in FIG. 4.

FIG. 6 is a plot of mass loss rates for a batch of avocados that were coated and then subsequently had their coatings removed by soaking in substantially pure ethanol at 22° C. for different amounts of time.

FIG. 7 is a plot of mass loss rates for a batch of avocados that were coated and subsequently had their coatings removed by soaking in substantially pure ethanol for ten seconds at varying temperatures.

FIG. 8 is a plot of the percent area reduction as a function of time for the avocados measured in FIG. 7.

FIG. 9 shows a flow chart diagramming a process for controlling or delaying ripening in produce by forming a coating over the produce and subsequently modifying the coating.

FIG. 10 is a plot of mass loss rates for finger limes that were coated with a formulation comprising 1,3-dihydroxypropan-2-yl palmitate and oleic acid 2-glycerol.

FIG. 11 is a plot of mass loss rates for finger limes that were coated with a formulation comprising 1,3-dihydroxypropan-2-yl palmitate and oleic acid.

FIG. 12 is a plot of respiration rate of avocados as a function of time removed from cold storage.

FIG. 13 is a plot of stiffness as measured by a durometer, of avocados as a function of time removed from cold storage.

### DETAILED DESCRIPTION

Many types of produce and other agricultural products (e.g., fruits, vegetables, roots, tubers, flowers) are harvested prior to complete ripening and then allowed to fully ripen post-harvest, for example during storage or shipping. While the practice of early harvesting generally necessitates storing the produce for longer periods of time after harvesting and before consumption, it also increases the amount of time between harvesting and spoilage of the produce, thereby allowing the produce to be shipped to more remote locations and be more widely distributed than would otherwise be possible if it were harvested closer to complete ripening. Furthermore, some types of produce never fully ripen prior to harvesting, and therefore should be stored post-harvest for at least some amount of time prior to consumption. In many cases, produce which has been harvested prior to complete ripening is subsequently treated with a ripening agent, for example ethylene gas, in order to increase the rate of ripening. However, in the case of seasonal products, it is still the case that there can be an oversupply of the products during the peak of the season, while after the season ends the products become unavailable or in some cases must be imported from remote locations.

Described herein are formulations and methods for delaying the initiation of ripening and for controlling the mass loss rate and/or the rate of ripening of agricultural products such as harvested produce. The methods generally include first forming a protective coating over the products in order to prevent ripening agents such as oxygen or ethylene from being adsorbed into the products, and/or to reduce the rate of moisture loss from the products. The protective coating can, for example, be formed from a composition of monomers, oligomers, low molecular weight polymers, or combinations thereof disposed over an outer surface of the agricultural products, and can be formulated to reduce the rate of post-harvest ripening. The protective coating can be maintained over the products for a time sufficient to delay the ripening of the products, after which the coating is removed or otherwise modified to reduce its efficacy and the products allowed to ripen. By staggering the times at which coatings formed over a batch of harvested products are formed and/or removed or modified, ripened products from the batch can be incrementally provided to consumers over extended periods of time.

Produce is generally deemed to be ripe when it is in a state of maturity such that a consumer would consider it fit for consumption. Produce typically ages/matures until a point at which it is considered by a consumer to be ripe and remains ripe for a period of time while continuing to age. If the produce is not consumed while ripe, it eventually spoils and is no longer fit for consumption. A number of factors, such as color, texture, and firmness (or softness), figure into a consumer's determination of when an agricultural product is ripe as well as when the agricultural product is spoiled. These various ripeness determination factors are weighted differently by consumers for different agricultural products. For example, in the case of tomatoes, color (e.g., how red the tomato appears and the corresponding color index of the tomato) is typically the most important factor in a consumer's determination of ripeness, while firmness and/or skin texture is typically used by a consumer to determine when a tomato has spoiled.

As used herein, “climacteric respiration” is understood to mean an increased level of cellular respiration associated with increased ethylene production and stage of ripening for certain species of produce.

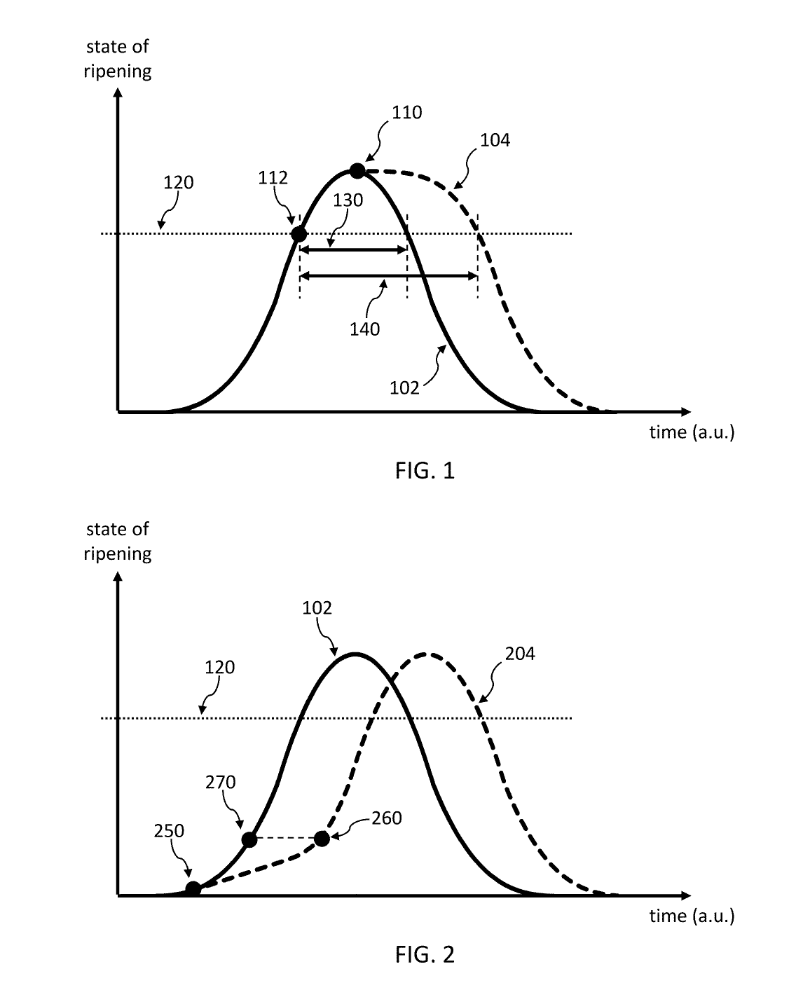

FIG. 1 is a qualitative plot indicating the relative state of ripening of produce as a function of time, where curve 102 represents the typical ripening cycle of produce harvested at a time corresponding to point 112, which is the onset of when the product is considered ripe. Dotted line 120 represents the relative level of ripening (e.g., the relative state of one or more factors used by a consumer to determine whether the produce is ripe) at which a consumer would consider the produce to be ripe. Prior to reaching the state of being ripe, the produce ages and matures until the time corresponding to point 112, after which it is considered to have ripened. As the produce continues to age, its state of ripening eventually falls below line 120 again, at which point it is considered to be spoiled. In some cases, the produce can be harvested after it has ripened (i.e., after the time represented by point 112), whereas in other cases it can be harvested prior to ripening (i.e., before the time represented by point 112). In the case that the produce is harvested precisely at the time represented by point 112, segment 130 represents the total shelf life of the produce.

In some embodiments, the protective coatings described herein can be used to extend the shelf life of the produce. For example, if a coating is applied at the time represented by point 110 in FIG. 1 to produce harvested at the time represented by point 112, the factors which determine when the produce has spoiled can progress at a slower rate, and the relative state of ripeness of the produce can follow dashed curve 104. Thus, by applying the coating, the shelf life of the produce can be extended from the time represented by segment 130 to the time represented by segment 140.

In some embodiments, the protective coatings described herein can be used to delay and/or control ripening of the produce. For example, referring now to FIG. 2, if a protective coating is applied to the produce prior to ripening, such as at the time represented by point 250, the ripening cycle of the produce proceeds along dashed curve 204 rather than curve 102, thereby delaying the onset of the produce being ripe. In some cases, the coating can be removed or otherwise modified to decrease its efficacy, for example at point 260 in FIG. 2. As shown in FIG. 2, after the coating is removed (at point 260), the ripening curve 204 of the produce can proceed similarly to the ripening curve 102 of uncoated produce which is at the same stage of ripening (i.e., at point 270). The above relationships can hold true when the coating is applied (and optionally removed) prior to harvesting of the produce, and also when the coating is applied and removed after harvesting of the produce.

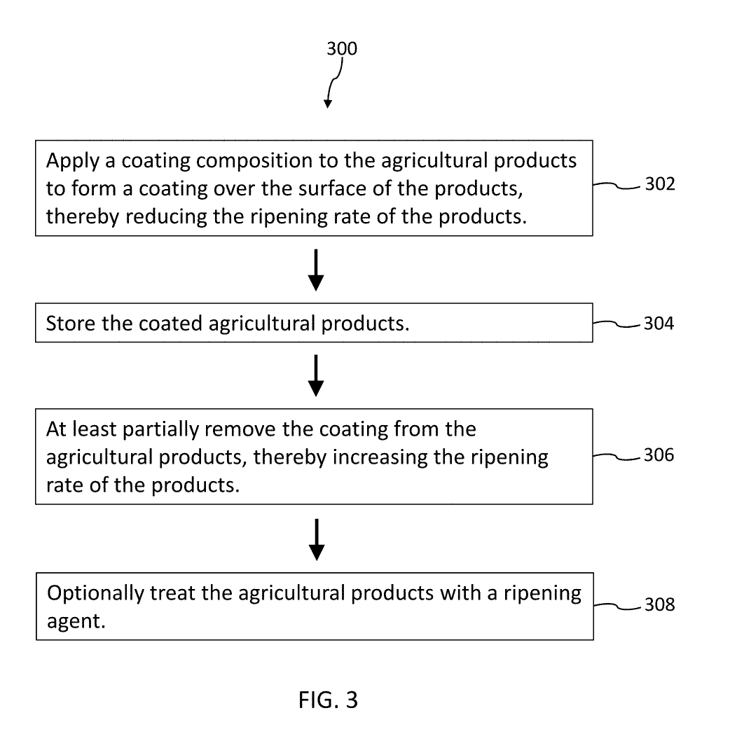

A method 300 of treating/storing produce and/or controlling or delaying the rate of ripening in harvested produce and/or other agricultural products is illustrated in FIG. 3. First, a coating composition is applied to the products to form a coating over a surface of the products (step 302). The coating composition can, for example, include a plurality of monomers, oligomers, low molecular weight polymers, or combinations thereof. In some embodiments, the coating composition is dissolved in a solvent to form a solution, the solution is applied to the agricultural products, and the solvent is then allowed to at least partially evaporate, thereby resulting in a coating comprising the constituents of the coating composition being formed over the surface of the products. The coating can be applied either before or after harvesting of the agricultural products.

Next, the coated agricultural products are stored (step 304), for example during sorting, processing, and/or shipping. In cases where the coating is applied prior to harvesting, the agricultural product can be harvested before storage. As described in detail below, the coating composition is formulated such that the coating causes a reduction in the rate of ripening of the agricultural products relative to what the rate of ripening would have been in the absence of the coating. For example, the coating composition can be formulated such that the coating serves as a barrier to oxidation and/or other ripening agents (e.g., ethylene). The coating composition may also be formulated such that the coating serves as a barrier to moisture, thereby reducing the rate of mass loss of the agricultural products. The coating composition may also be formulated such that the coating causes a reduction in the respiration rate of the agricultural products. Consequently, the agricultural products ripen and optionally lose mass at a reduced rate during storage, thereby delaying ripening and prolonging the life of the harvested products. For example, prior to coating the products, the products may ripen at a first average rate, whereas after the coating is formed the products may ripen at a second average rate which is less than the first average rate.

Finally, when full ripening of the agricultural products is desired, the coating is at least partially removed, for example by soaking the products in a solvent that dissolves the coating (step 306). In some embodiments, substantially all of the coating is removed. Removing (or at least partially removing) the coating causes the rate of ripening of the agricultural products to increase relative to the rate of ripening while the products have the coatings thereon. For example, during the time that the coatings are on the products, the products can ripen at the second average rate described above, whereas after the coatings are removed the products can ripen at a third average rate which is greater than the second average rate. In this way, ripening of the products can be delayed and/or controlled.

As also shown in FIG. 3, after removal (or at least partial removal) of the coatings, the agricultural products may optionally be treated with a ripening agent in order to further accelerate ripening (step 308). For example, the products can be subjected to ethylene gas to accelerate ripening.

Alternatively, as set forth herein and described in further detail below, the coatings can be left on the agricultural products, and can optionally be modified to reduce their efficacy in slowing or delaying ripening. The coatings described herein can also be substantially tasteless, colorless, and/or odorless. Moreover, the coatings described herein can be safe for human consumption. Accordingly, in some embodiments the coatings described herein are substantially not removed from the agricultural product and can be eaten by a consumer.

The process steps 302, 304, 306, and 308 of process 300 (FIG. 3) and their associated processing agents and resultant coatings are now described in further detail. Referring to step 302, forming a coating over the surface of the agricultural product can, for example, be achieved by the following steps. First, a solid mixture of a coating agent (e.g., a composition of monomer and/or oligomer, and/or polymer units) is dissolved in a solvent (e.g., ethanol, methanol, acetone, isopropanol, ethyl acetate, water, or combinations thereof) to form a solution. The concentration of the coating agent in the solvent can, for example, be in a range of about 0.1 to 200 mg/mL. Next, the solution, which includes the coating agent, is applied over the surface of the produce or other agricultural product to be coated, for example by spray coating the produce/product or by dipping the produce/product in the solution. In the case of spray coating, the solution can, for example, be placed in a spray bottle that generates a fine mist spray. The spray bottle head can then be held approximately three to twelve inches from the produce/product, and the produce/product then sprayed. In the case of dip coating, the produce/product can, for example, be placed in a bag, the solution containing the coating agent poured into the bag, and the bag then sealed and its contents lightly tumbled or agitated until the entire surface of the produce/product is wet. After applying the solution to the produce/product, the produce/product is allowed to dry until the solvent has at least partially evaporated, thereby allowing a protective coating composed of the constituents of the coating agent (e.g., monomer and/or oligomer and/or polymer units) to form over the surface of the produce/product.

The coating agent that is dissolved in the solvent can include a plurality of monomers, oligomers, polymers (e.g., low molecular weight polymers), or combinations thereof. The specific composition of monomers, oligomers, polymers, or combinations thereof can be formulated such that the resulting coating formed over the agricultural product mimics or enhances the cuticular layer of the product. The biopolyester cutin forms the main structural component of the cuticle that composes the aerial surface of most land plants. Cutin is formed from a mixture of polymerized mono- and/or polyhydroxy fatty acids and esters, as well as embedded cuticular waxes. The hydroxy fatty acids and esters of the cuticle layer form tightly bound networks with high crosslink density, thereby acting as a barrier to moisture loss and oxidation, as well as providing protection against other environmental stressors.

The monomers, oligomers, polymers, or combinations thereof from which the coating agent is comprised can be extracted or derived from plant matter, and in particular from cutin obtained from plant matter. Plant matter typically includes some portions that contain cutin and/or have a high density of cutin (e.g., fruit peels, leaves, shoots, etc.), as well as other portions that do not contain cutin or have a low density of cutin (e.g., fruit flesh, seeds, etc.). The cutin-containing portions can be formed from the monomer and/or oligomer and/or polymer units that are subsequently utilized in the formulations described herein for forming the coatings over the surface of the agricultural products. The cutin-containing portions can also include other constituents such as non-hydroxylated fatty acids and esters, proteins, polysaccharides, phenols, lignans, aromatic acids, terpenoids, flavonoids, carotenoids, alkaloids, alcohols, alkanes, and aldehydes, which may be included in the formulations or may be omitted.

The monomers, oligomers, polymers, or combinations thereof can be obtained by first separating (or at least partially separating) portions of the plant that include molecules desirable for the coating agents from those that do not include the desired molecules. For example, when utilizing cutin as the feedstock for the coating agent composition, the cutin-containing portions of the plant matter are separated (or at least partially separated) from non-cutin-containing portions, and cutin is obtained from the cutin-containing portions (e.g., when the cutin-containing portion is a fruit peel, the cutin is separated from the peel). The obtained portion of the plant (e.g., cutin) is then depolymerized (or at least partially depolymerized) in order to obtain a mixture including a plurality of fatty acid or esterified cutin monomers, oligomers, polymers (e.g., low molecular weight polymers), or combinations thereof.

A method of obtaining fatty acid or esterified cutin-derived monomers, oligomers, polymers, or combinations thereof can include (i) obtaining cutin from a cutin-containing portion of a plant matter, the cutin-containing portion being at least partially separated from a non-cutin-containing portion of the plant matter; (ii) at least partially depolymerizing the cutin in a first solvent to obtain a first solution comprising a first intermediate extract dissolved in the first solvent, the first solution having a pH in the range of 10 to 14, the first intermediate extract including a plurality of cutin-derived monomers, oligomers, or combinations thereof; (iii) evaporating at least a portion of the first solvent, causing the first intermediate extract to solidify; (iv) dissolving the solidified first intermediate extract in a polar solvent to obtain a second solution; and (v) acidifying the second solution, causing the first intermediate extract to resolidify.

Another method of obtaining fatty acid or esterified cutin-derived monomers, oligomers, polymers, or combinations thereof can include (i) obtaining cutin from a cutin-containing portion of a plant matter, the cutin-containing portion being at least partially separated from a non-cutin-containing portion of the plant matter; (ii) at least partially depolymerizing the cutin in a first solvent to obtain a first solution comprising a first intermediate extract dissolved in the first solvent, the first intermediate extract including a plurality of cutin-derived monomers, oligomers, or combinations thereof; (iii) acidifying the first intermediate extract; (iv) selectively filtering the first intermediate extract to obtain a second intermediate extract having a higher purity than the first intermediate extract; and (v) dissolving the second intermediate extract in a second solvent to obtain the desired molecules.

Another method of obtaining fatty acid or esterified cutin-derived monomers, oligomers, polymers, or combinations thereof can include (i) obtaining cutin from a cutin-containing portion of a plant matter, the cutin-containing portion being at least partially separated from a non-cutin-containing portion of the plant matter; (ii) at least partially depolymerizing the cutin in a first solvent to obtain a first solution comprising a first intermediate extract in the first solvent, the first intermediate extract including a plurality of cutin-derived monomers, oligomers, or combinations thereof; (iii) selectively filtering the first intermediate extract to obtain a second intermediate extract having a higher purity than the first intermediate extract, the second intermediate extract including at least one of the cutin-derived monomers, oligomers, or combinations thereof; and (iv) functionalizing the cutin-derived monomers, oligomers, or combinations thereof of the second intermediate extract (for example by esterification, such as esterification with a glycerol molecule) to obtain the desired molecules.

Another method of obtaining fatty acid or esterified cutin-derived monomers, oligomers, polymers, or combinations thereof can include (i) obtaining cutin from a cutin-containing portion of a plant matter, the cutin-containing portion being at least partially separated from a non-cutin-containing portion of the plant matter; (ii) at least partially depolymerizing the cutin in a first solvent to obtain a first solution comprising a first intermediate extract dissolved in the first solvent, the first solution having a pH in the range of 10 to 14, the first intermediate extract including a plurality of cutin-derived monomers, oligomers, or combinations thereof; (iii) evaporating at least 25% of a volume of the first solvent from the first solution; (iv) adding a polar solvent to the first solution, thereby obtaining a second solution; and (v) acidifying the second solution, thereby causing the first intermediate extract to precipitate.

Another method of obtaining fatty acid or esterified cutin-derived monomers, oligomers, polymers, or combinations thereof can include (i) obtaining cutin from cutin-containing plant matter; (ii) adding the cutin to a solvent to form a first mixture, the solvent having a boiling point at a first temperature at a pressure of one atmosphere; and (iii) heating the first mixture to a second temperature and second pressure, the second temperature being higher than the first temperature and the second pressure being higher than one atmosphere, to form a second mixture comprising the cutin-derived monomers, oligomers, or combinations thereof. For example, the solvent can be water, and the first temperature can be about 100° C.

Another method of preparing a composition comprising cutin-derived free fatty acid monomers, oligomers, or combinations thereof can include (i) obtaining cutin from cutin-containing plant matter; (ii) adding the cutin to water to form a mixture; and (iii) heating the mixture from a first temperature and first pressure to a second temperature and second pressure, the second temperature being higher than the boiling point of water at one atmosphere and the second pressure being higher than one atmosphere, thereby forming the composition comprising the cutin-derived free fatty acid monomers, oligomers, or combinations thereof.

Another method of preparing a composition comprising esters of cutin-derived fatty acids can include (i) obtaining cutin from cutin-containing plant matter; (ii) adding the cutin to a solvent to form a mixture, the solvent having a boiling point at a first temperature at a pressure of one atmosphere; and (iii) heating the mixture to a second temperature and second pressure, the second temperature being higher than the first temperature and the second pressure being higher than one atmosphere, thereby forming the composition comprising the esters. In some implementations, the solvent comprises methanol and the esters comprise methyl esters. In some implementations, the solvent comprises ethanol and the esters comprise ethyl esters. In some implementations, the solvent comprises glycerol and the esters comprise glyceryl esters.

Another method of preparing a composition comprising fatty acid esters can include (i) providing a crosslinked polyester (e.g., cutin) comprising fatty acids; (ii) treating the polyester with an acid and an alcohol; and (iii) removing the acid and alcohol to isolate the resulting fatty acid esters.

Additional methods of obtaining fatty acid or esterified cutin derived monomers, oligomers, polymers, or combinations thereof are described in International Patent Application No. PCT/US16/33617, entitled “PLANT EXTRACT COMPOSITIONS AND METHODS OF PREPARATION THEREOF,” filed on May 20, 2016, International Patent Application No. PCT/US16/65917, entitled “PLANT EXTRACT COMPOSITIONS FOR FORMING PROTECTIVE COATINGS,” filed on Dec. 9, 2016, and U.S. Provisional Patent Application No. 62/423,337, entitled “COMPOSITIONS FORMED FROM PLANT EXTRACTS AND METHODS OF PREPARATION THEREOF,” filed on Nov. 17, 2016, the disclosures of which are incorporated herein by reference in their entirety.

The cutin derived monomers, oligomers, polymers, or combinations thereof can be directly dissolved in the solvent to form the solution used in the formation of the coatings, or alternatively can first be activated or chemically modified (e.g., functionalized). Chemical modification or activation can, for example, include glycerating the monomers, oligomers, polymers, or combinations thereof to form a mixture of 1-monoacylglycerides and/or 2-monoacylglycerides, and the mixture of 1-monoacylglycerides and/or 2-monoacylglycerides is dissolved in the solvent to form a solution, thereby resulting in the formulation utilized for preparation of the protective coating. As used herein, functionalized monomers, oligomers or polymers are monomers, oligomers, or polymers for which the chemical composition has been modified (e.g., by attaching a functional group such as glyceryl).

In some implementations, the coating agent comprises fatty acids, esters, triglycerides, diglycerides, monoglycerides (e.g., monoacylglycerides), amides, amines, thiols, thioesters, carboxylic acids, ethers, aliphatic waxes, alcohols, salts (inorganic and organic), acids, bases, proteins, enzymes, or combinations thereof. In some implementations, the coating agent can be substantially similar to or the same as those described in U.S. patent application Ser. No. 15/330,403 entitled “Precursor Compounds for Molecular Coatings,” filed on Sep. 15, 2016, the disclosure of which is incorporated herein by reference in its entirety. In some implementations, the coating agent comprises monoacylglycerides (e.g., 1-monoacylglycerides or 2-monoacylglycerides), for example monoacylglyceride monomers and/or oligomers and/or low molecular weight polymers formed thereof. For example, the coating agent can include compounds of Formula I:

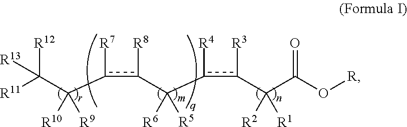

wherein:

R is selected from —H, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl or heteroaryl is optionally substituted with one or more C₁-C₆ alkyl or hydroxy;

R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;

R³, R⁴, R⁷ and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, —SR¹⁴, or halogen; or

R³ and R⁴ can combine with the carbon atoms to which they are attached to form a C₃-C₆ cycloalkyl, a C₄-C₆ cycloalkenyl, or 3 to 6-membered ring, heterocycle; and/or

R⁷ and R⁸ can combine with the carbon atoms to which they are attached to form a C₃-C₆ cycloalkyl, a C₄-C₆ cycloalkenyl, or 3 to 6-membered ring, heterocycle; and/or

R¹⁴ and R¹⁵ are each independently, at each occurrence, —H, —C₁-C₆ alkyl, —C₂-C₆ alkenyl, or —C₂-C₆ alkynyl;

the symbol [symbol] represents an optionally single or cis or trans double bond;

n is 0, 1, 2, 3, 4, 5, 6, 7, or 8;

m is 0, 1, 2, or 3;

q is 0, 1, 2, 3, 4, or 5; and

r is 0, 1, 2, 3, 4, 5, 6, 7, or 8.

In some implementations, R is —H, —CH₃, or —CH₂CH₃.

In some implementations, the coating agent comprises monoacylglyceride (e.g., 1-monoacylglyceride or 2-monoacylglyceride) esters and/or monomers and/or oligomers and/or low molecular weight polymers formed thereof. The difference between a 1-monoacylglyceride and a 2-monoacylglyceride is the point of connection of the glycerol ester. Accordingly, in some implementations, the coating agent includes compounds of Formula I-A (e.g., 2-monoacylglycerides):

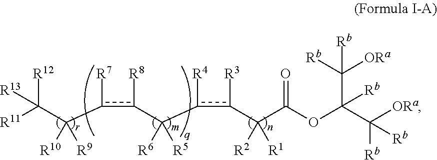

wherein:

each R^{a} is independently —H or —C₁-C₆ alkyl;

each R^{b} is independently selected from —H, —C₁-C₆ alkyl, or —OH;

R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;

R³, R⁴, R⁷ and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, —SR¹⁴, or halogen; or

R³ and R⁴ can combine with the carbon atoms to which they are attached to form a C₃-C₆ cycloalkyl, a C₄-C₆ cycloalkenyl, or 3 to 6-membered ring, heterocycle; and/or

R⁷ and R⁸ can combine with the carbon atoms to which they are attached to form a C₃-C₆ cycloalkyl, a C₄-C₆ cycloalkenyl, or 3 to 6-membered ring, heterocycle; and/or

R¹⁴ and R¹⁵ are each independently, at each occurrence, —H, —C₁-C₆ alkyl, —C₂-C₆ alkenyl, or —C₂-C₆ alkynyl;

the symbol [symbol] represents an optionally single or cis or trans double bond;

n is 0, 1, 2, 3, 4, 5, 6, 7, or 8;

m is 0, 1, 2, or 3;

q is 0, 1, 2, 3, 4, or 5; and

r is 0, 1, 2, 3, 4, 5, 6, 7, or 8.

In some implementations, the coating agent includes compounds of Formula I-B (e.g., 1-monoacylglycerides):

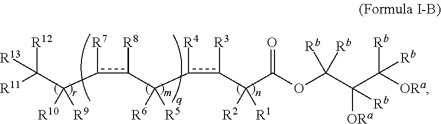

wherein:

each R^{a} is independently —H or —C₁-C₆ alkyl;

each R^{b} is independently selected from —H, —C₁-C₆ alkyl, or —OH;

R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR₁₄, or halogen;

R³, R⁴, R⁷ and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR₁₄, —NR¹⁴R¹⁵, —SR¹⁴ or halogen; or

R³ and R⁴ can combine with the carbon atoms to which they are attached to form a C₃-C₆ cycloalkyl, a C₄-C₆ cycloalkenyl, or 3 to 6-membered ring, heterocycle; and/or

R⁷ and R⁸ can combine with the carbon atoms to which they are attached to form a C₃-C₆ cycloalkyl, a C₄-C₆ cycloalkenyl, or 3 to 6-membered ring, heterocycle; and/or

R¹⁴ and R¹⁵ are each independently, at each occurrence, —H, —C₁-C₆ alkyl, —C₂-C₆ alkenyl, or —C₂-C₆ alkynyl;

the symbol [symbol] represents an optionally single or cis or trans double bond;

n is 0, 1, 2, 3, 4, 5, 6, 7, or 8;

m is 0, 1, 2, or 3;

q is 0, 1, 2, 3, 4, or 5; and

r is 0, 1, 2, 3, 4, 5, 6, 7, or 8.

In some embodiments of Formula I-A and/or Formula I-B, a 2-glyceryl or a 1-glyceryl is optionally substituted with one or more of —H, C₁-C₆ alkyl, or hydroxy. In one or more embodiments, a mass ratio of the compound of Formula I-B to the compound of Formula I-A in the coating agent is in a range of 0.1 to 1.0.

In some embodiments, the coating agent includes one or more of the following fatty acid compounds:

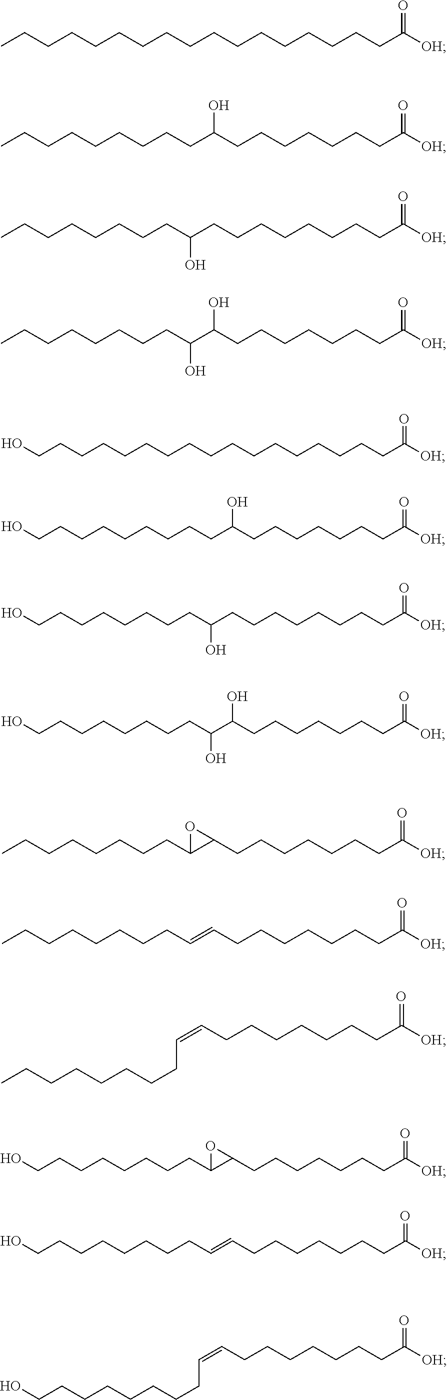

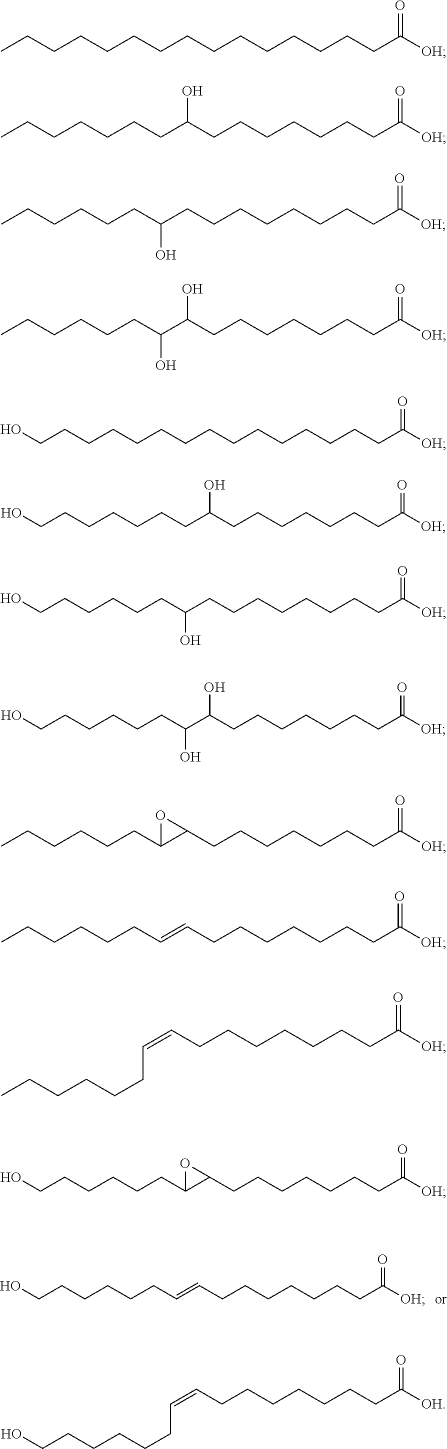

In some embodiments, the coating agent includes one or more of the following methyl ester compounds:

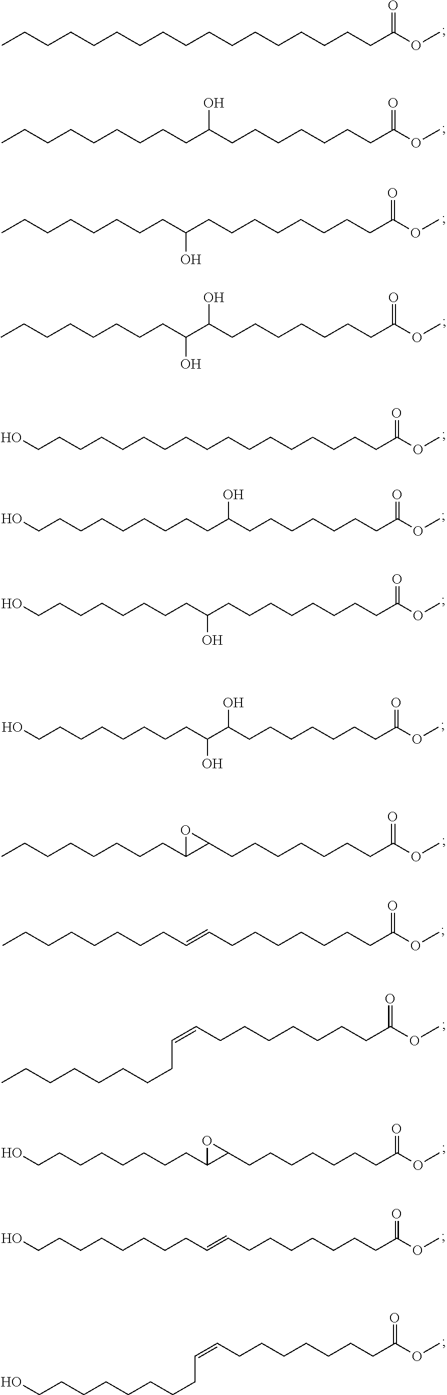

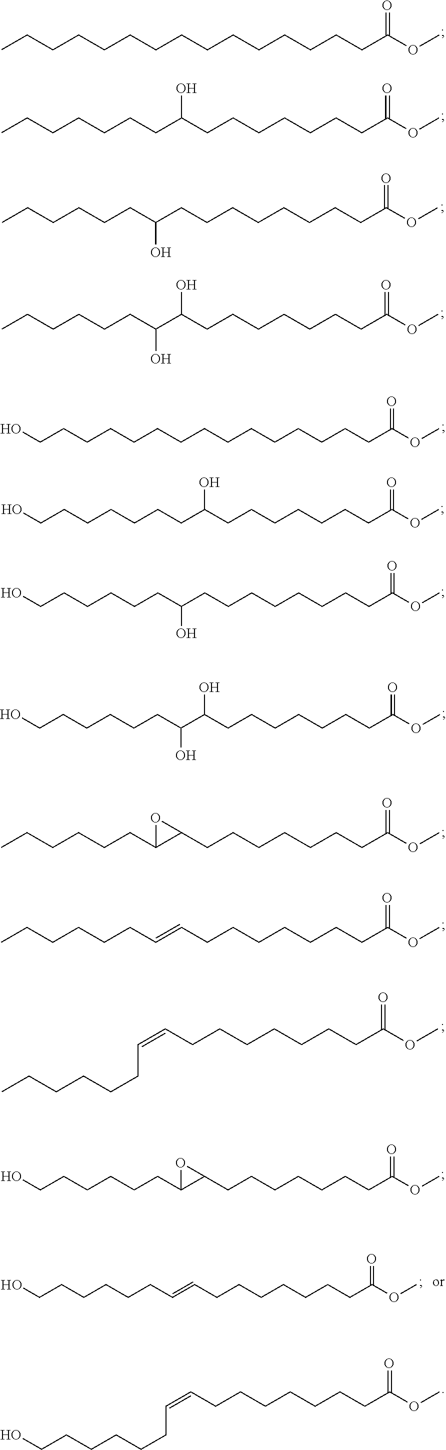

In some embodiments, the coating agent includes one or more of the following ethyl ester compounds:

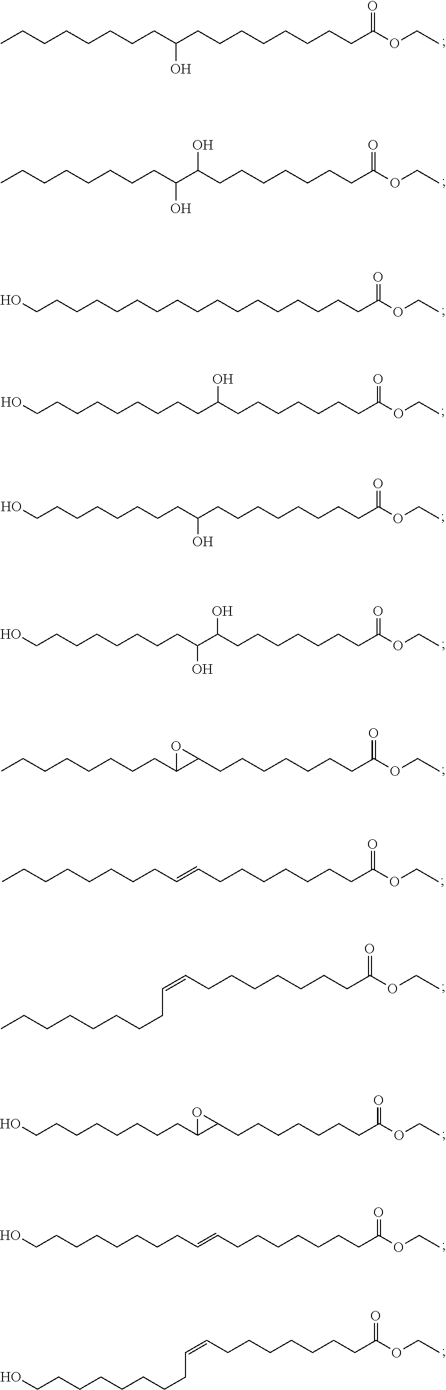

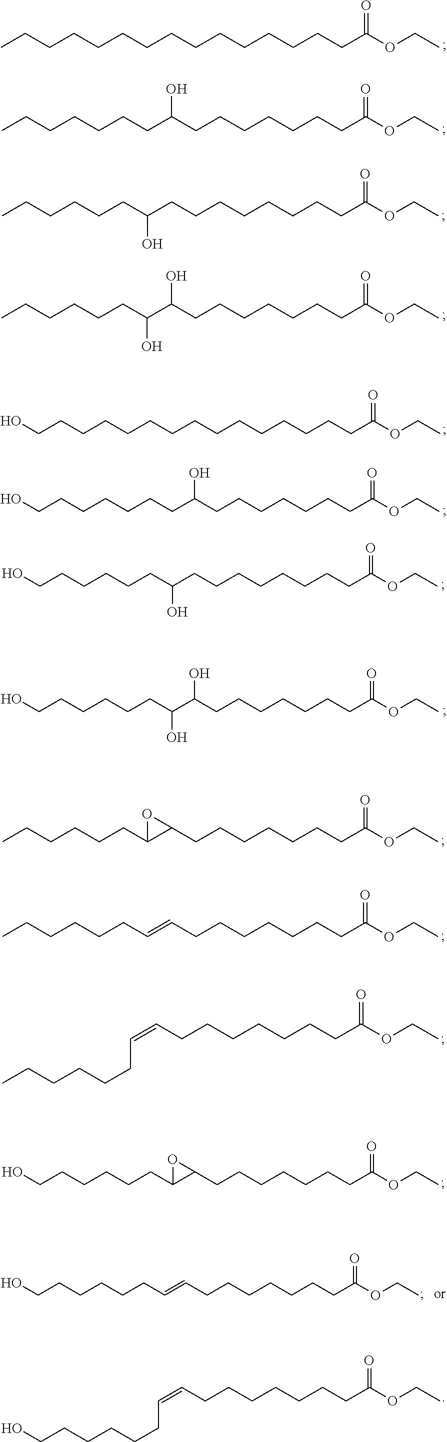

In some embodiments, the coating agent includes one or more of the following 2-glycerol ester compounds:

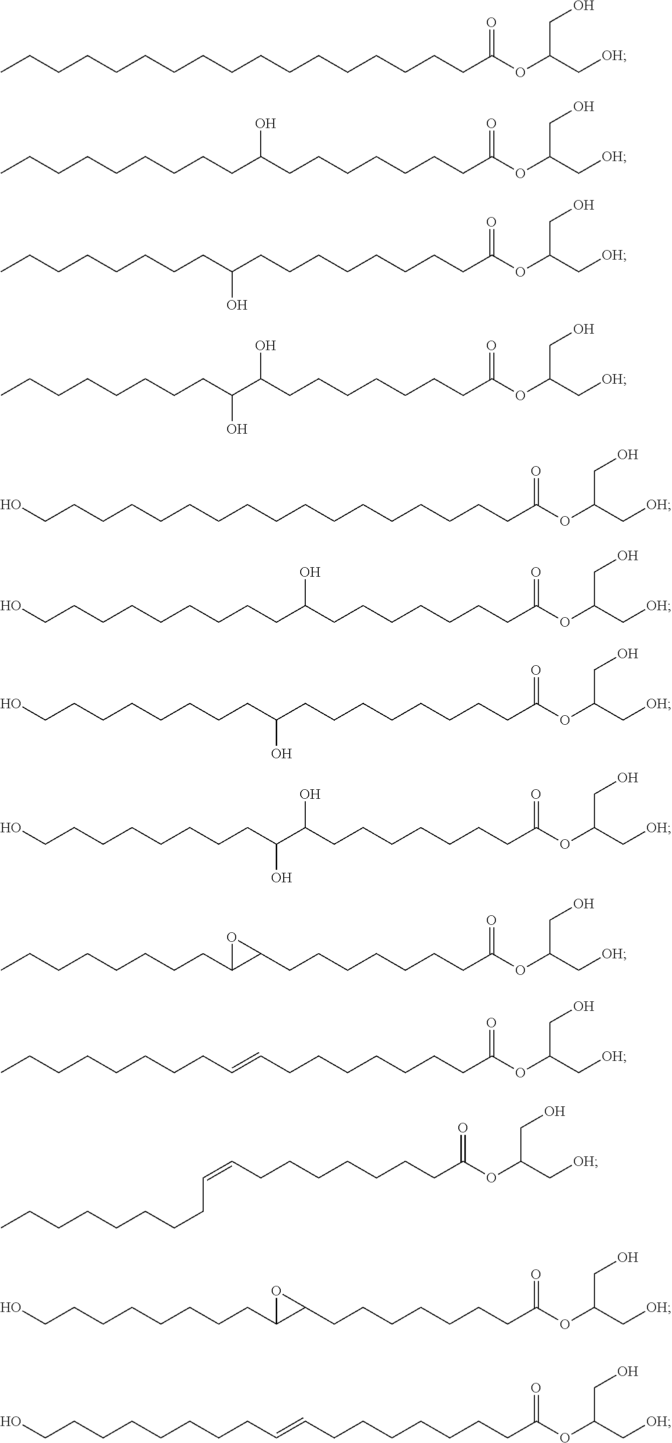

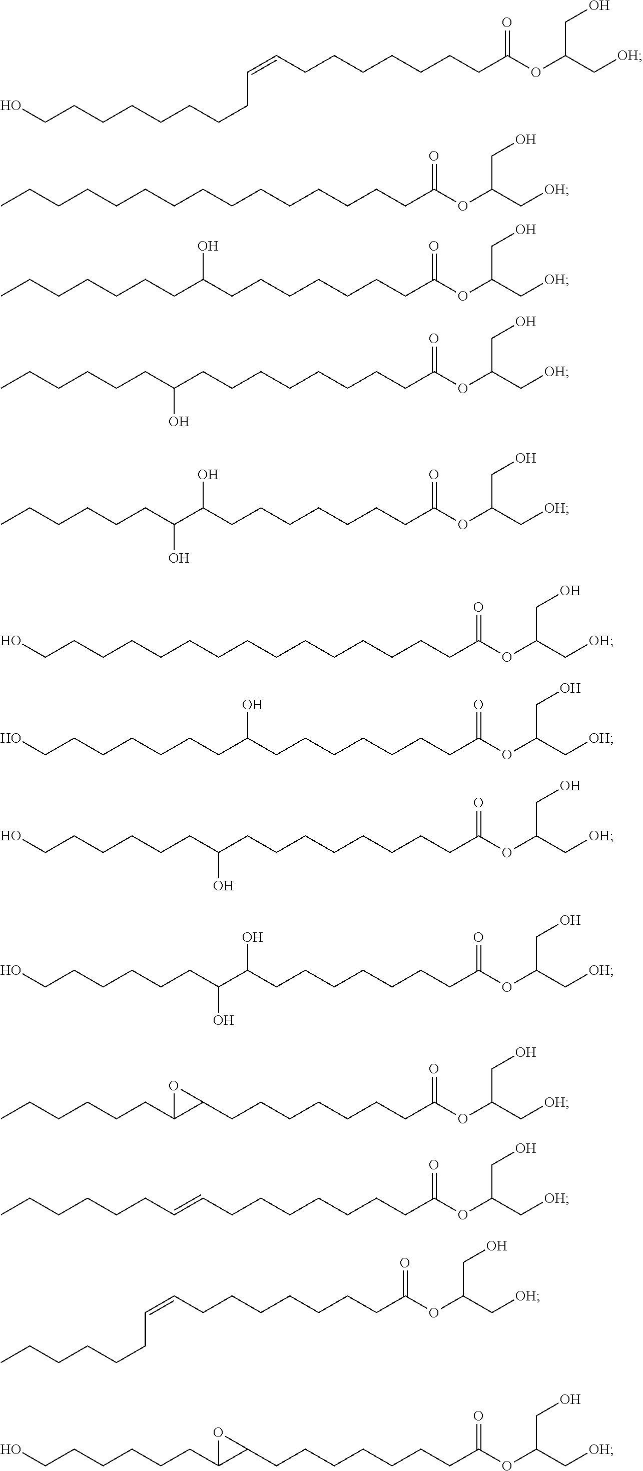

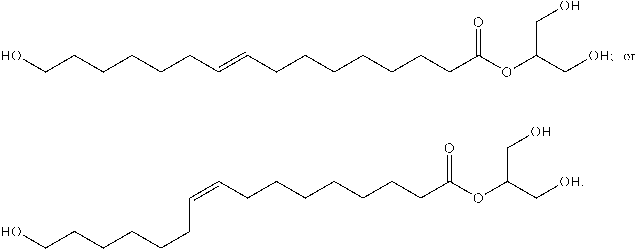 [SMILES: O=C(CCCCCCC/C=C/CCCCCCO)OC(CO)CO ; O=C(CCCCCCC/C=C\CCCCCCO)OC(CO)CO]

In some embodiments, the coating agent includes one or more of the following 1-glycerol ester compounds:

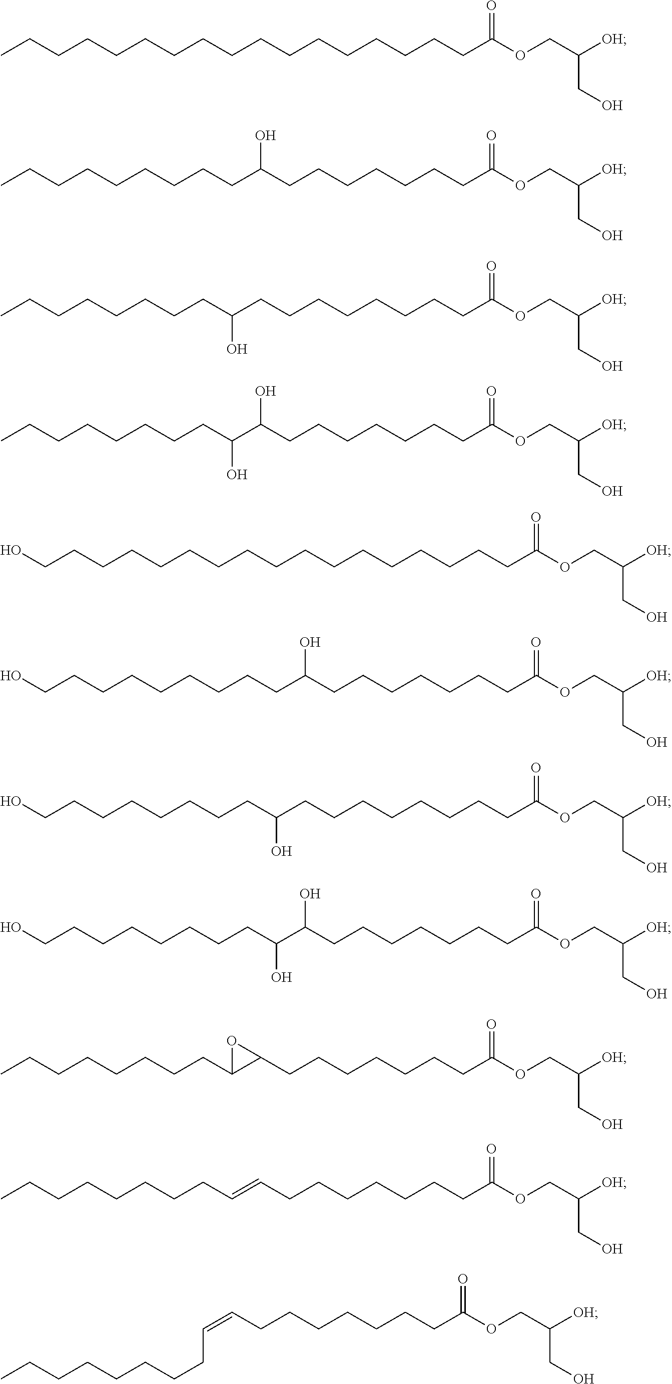

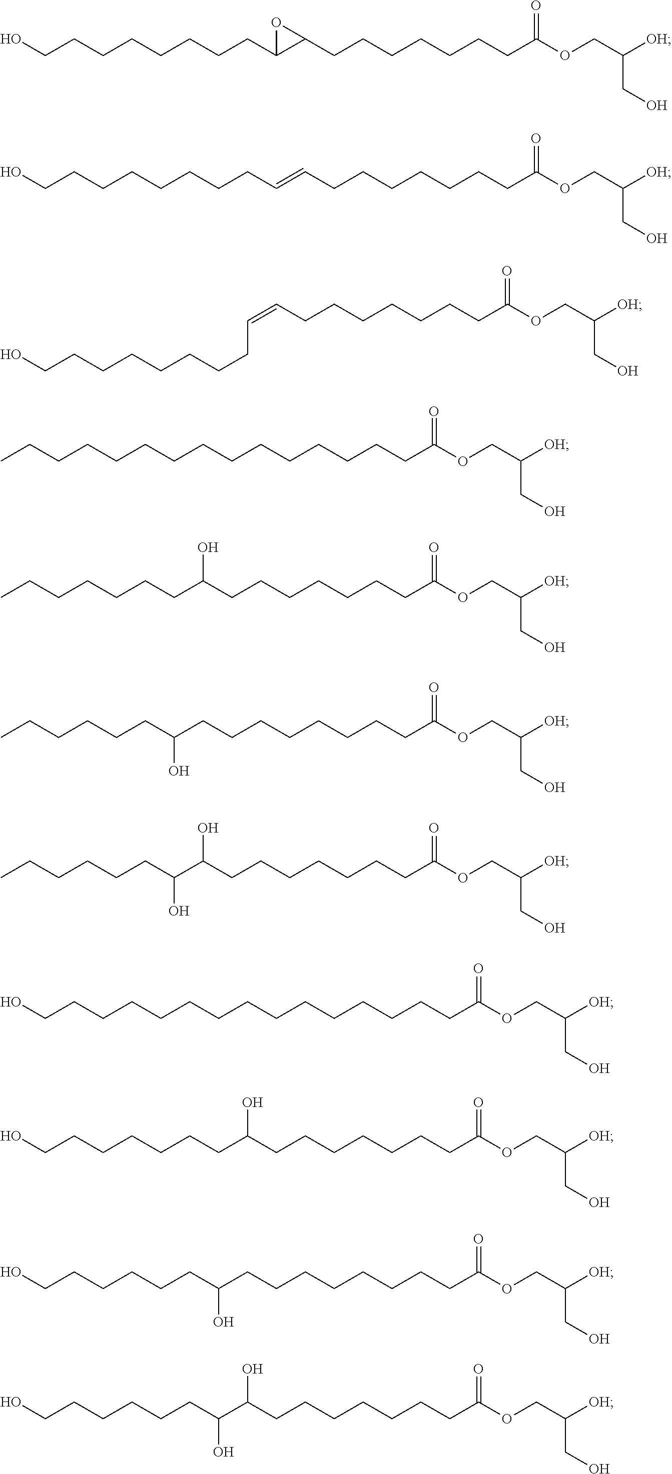

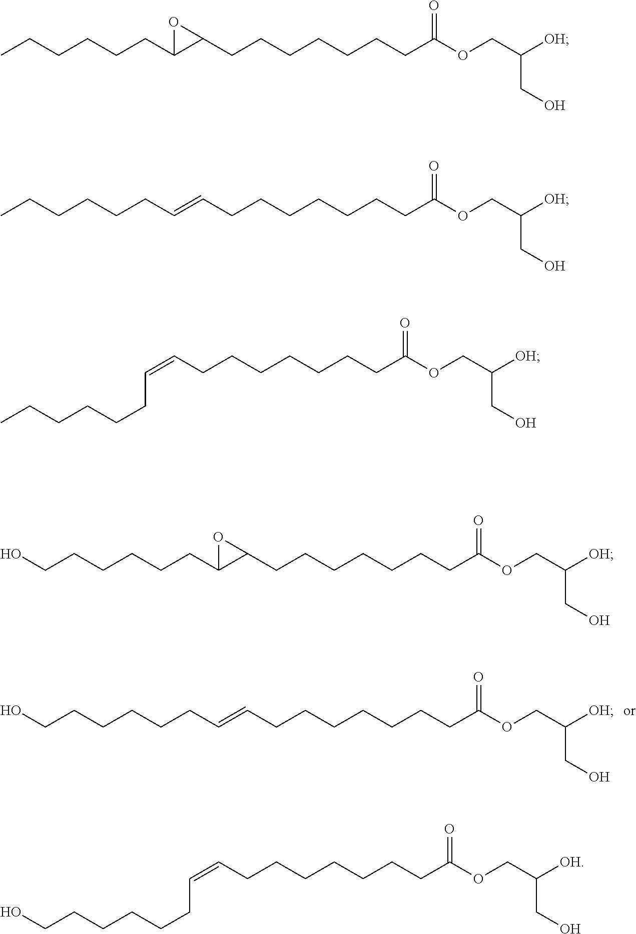 [SMILES: CCCCCCC1OC1CCCCCCCC(=O)OCC(O)CO ; CCCCCC/C=C/CCCCCCCC(=O)OCC(O)CO ; CCCCCC/C=C\CCCCCCCC(=O)OCC(O)CO ; O=C(CCCCCCCC1OC1CCCCCCO)OCC(O)CO ; O=C(CCCCCCC/C=C/CCCCCCO)OCC(O)CO ; O=C(CCCCCCC/C=C\CCCCCCO)OCC(O)CO]

In some embodiments, the coating agent is formed of a combination of at least 2 different compounds. For example, the coating agent can comprise a compound of Formula I-A and an additive. The additive can, for example, include a saturated or unsaturated compound of Formula I-B, a saturated or unsaturated fatty acid, an ethyl ester, or a second compound of Formula I-A which is different from the (first) compound of Formula I-A (e.g., has a different length carbon chain). The compound of Formula I-A can make up at least 10%, at least 20%, at least 30%, at least 40%, at least 50%, at least 60%, at least 70%, at least 80%, or at least 90% of the mass of the coating agent. A combined mass of the compound of Formula I-A and the additive can be at least 10%, at least 20%, at least 30%, at least 40%, at least 50%, at least 60%, at least 70%, at least 80%, or at least 90% of the total mass of the coating agent. A molar ratio of the additive to the compound of Formula I-A in the coating agent can be in a range of 0.1 to 5, for example in a range of 0.1 to 4, 0.1 to 3, 0.1 to 2, 0.1 to 1, 0.1 to 0.9, 0.1 to 0.8, 0.1 to 0.7, 0.1 to 0.6, 0.1 to 0.5, 0.15 to 5, 0.15 to 4, 0.15 to 3, 0.15 to 2, 0.15 to 1, 0.15 to 0.9, 0.15 to 0.8, 0.15 to 0.7, 0.15 to 0.6, 0.15 to 0.5, 0.2 to 5, 0.2 to 4, 0.2 to 3, 0.2 to 2, 0.2 to 1, 0.2 to 0.9, 0.2 to 0.8, 0.2 to 0.7, 0.2 to 0.6, 0.2 to 0.5, 0.3 to 5, 0.3 to 4, 0.3 to 3, 0.3 to 2, 0.3 to 1, 0.3 to 0.9, 0.3 to 0.8, 0.3 to 0.7, 0.3 to 0.6, 0.3 to 0.5, 1 to 5, 1 to 4, 1 to 3, or 1 to 2. The coating agent can, for example, be formed from one of the combinations of a compound of Formula I-A and an additive listed in Table 1 below.

TABLE 1
Exemplary Coating Agent Compositions

| Compound | | |
|---|---|---|
| of Formula | | |
| I-A | Additive | Note |
| SA-2G | SA-1G | Additive is a saturated compound of Formula II |
| | | (1-monoacylglyceride) with the same length |
| | | carbon chain as the compound of Formula I |
| PA-2G | PA-1G | Additive is a saturated compound of Formula II |
| | | (1-monoacylglyceride) with the same length |
| | | carbon chain as the compound of Formula I |
| PA-2G | MA-1G | Additive is a saturated compound of Formula II |
| | | (1-monoacylglyceride) with a shorter length |
| | | carbon chain than the compound of Formula I |
| PA-2G | OA-1G | Additive is an unsaturated compound of |
| | | Formula II (1-monoacylglyceride) with a longer |
| | | length carbon chain than the compound of |
| | | Formula I |
| PA-2G | SA-1G | Additive is a saturated compound of Formula II |
| | | (1-monoacylglyceride) with a longer length |
| | | carbon chain than the compound of Formula I |
| PA-2G | PA | Additive is a saturated fatty acid with the |
| | | same length carbon chain as the compound of |
| | | Formula I |
| PA-2G | OA | Additive is an unsaturated fatty acid with a |
| | | longer length carbon chain than the compound |
| | | of Formula I |
| PA-2G | SA | Additive is a saturated fatty acid with a |
| | | longer length carbon chain than the compound |
| | | of Formula I |
| PA-2G | MA | Additive is a saturated fatty acid with a |
| | | shorter length carbon chain than the compound |
| | | of Formula I |
| PA-2G | OA-2G | Additive is an unsaturated compound of |
| | | Formula I (2-monoacylglyceride) with a longer |
| | | carbon chain than PA-2G |
| PA-2G | EtPA | Additive is an ethyl ester. |

In some embodiments, the coating agent is formed from one of the combinations of compounds listed in Table 2 below.

TABLE 2
Exemplary Coating Agent Compositions

| | | (Optional) |
|---|---|---|
| Component 1 | Component 2 | Component 3 |
| SA-1G (Formula I-B) | MA (Fatty acid, shorter length carbon chain | |
| | than compound of Formula I-B) | |
| SA-1G (Formula I-B) | PA (Fatty acid, shorter length carbon chain | |
| | than compound of Formula I-B) | |
| SA-1G (Formula I-B) | SA (Fatty acid, same length carbon chain as | |
| | compound of Formula I-B) | |
| PA-1G (Formula I-B) | MA (Fatty acid, shorter length carbon chain | |
| | than compound of Formula I-B) | |
| PA-1G (Formula I-B) | PA (Fatty acid, same length carbon chain as | |
| | compound of Formula I-B) | |
| PA-1G (Formula I-B) | SA (Fatty acid, longer length carbon chain | |
| | than compound of Formula I-B) | |
| MA-1G (Formula I-B) | MA (Fatty acid, same length carbon chain | |
| | as compound of Formula I-B) | |
| MA-1G (Formula I-B) | PA (Fatty acid, longer length carbon chain | |
| | than compound of Formula I-B) | |
| MA-1G (Formula I-B) | SA (Fatty acid, longer length carbon chain | |
| | than compound of Formula I-B) | |
| SA-1G (First compound | PA-1G (Second compound of Formula II, | |
| of Formula I-B) | shorter carbon chain than First compound of | |
| | Formula I-B) | |
| SA-1G (First compound | MA-1G (Second compound of Formula II, | |
| of Formula I-B) | shorter carbon chain than First compound of | |
| | Formula I-B) | |
| MA-1G (First compound | PA-1G (Second compound of Formula II, | |
| of Formula I-B) | longer carbon chain than First compound of | |
| | Formula I-B) | |
| SA-1G (Formula I-B) | PA (Fatty acid, shorter length carbon chain | OA (Fatty acid, same |
| | than compound of Formula I-B) | length carbon chain as |
| | | compound of Formula I- |
| | | B) |

The following abbreviations are used in Tables 1 and 2 above. Hexadecanoic acid (i.e., palmitic acid) is abbreviated to PA. Octadecanoic acid (i.e., stearic acid) is abbreviated to SA. Tetradecanoic acid (i.e., myristic acid) is abbreviated to MA. (9Z)-Octadecenoic acid (i.e., oleic acid) is abbreviated to OA. 1,3-dihydroxypropan-2-yl palmitate (i.e., 2-glycero palmitate) is abbreviated to PA-2G. 1,3-dihydroxypropan-2-yl octadecanoate (i.e., 2-glycero stearate) is abbreviated to SA-2G. 1,3-dihydroxypropan-2-yl tetradecanoic acid (i.e., 2-glycero myristate) is abbreviated to MA-2G. 1,3-dihydroxypropan-2-yl (9Z)-Octadecenoate (i.e., 2-glycero oleate) is abbreviated to OA-2G. 2,3-dihydroxypropan-2-yl palmitate (i.e., 1-glycero palmitate) is abbreviated to PA-1G. 2,3-dihydroxypropan-2-yl octadecanoate (i.e., 1-glycero stearate) is abbreviated to SA-1G. 2,3-dihydroxypropan-2-yl tetradecanoate (i.e., 1-glycero myristate) is abbreviated to MA-1G. 2,3-dihydroxypropan-2-yl (9Z)-Octadecenoate (i.e., 1-glycero oleate) is abbreviated to OA-1G. Ethyl hexadecanoate (i.e., ethyl palmitate) is abbreviated to EtPA.

As seen in Table 2 above, the coating agent can include a first component and a second component, where the first component is a compound of Formula I-B and the second component is either a fatty acid or a second compound of Formula I-B which is different than the (first) compound of Formula I-B. The compound of Formula I-B can make up at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the mass of the coating agent. A combined mass of the first component and the second component can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% of the total mass of the coating agent.

Referring back to step 302 of process 300 (FIG. 3), after dissolving the coating agent in a solvent to form a solution, the solution is applied over the surface of a piece of produce or other agricultural product in order to form a protective coating over the surface, the protective coating being formed from constituents of the coating agent. As previously described, the solution can, for example, be applied to the surface by dipping the produce or agricultural product in the solution, or by spraying the solution over the surface. The solvent is then removed from the surface of the produce or agricultural product, for example by allowing the solvent to evaporate or at least partially evaporate. In some embodiments, the act of at least partially removing of the solvent from the surface of the produce can comprise removing at least 90% (e.g., at least 95%, at least 99%, or substantially all) of the solvent from the surface of the produce. As the solvent is removed (e.g., evaporated), the coating agent can re-solidify on the surface of the produce or agricultural product to form the protective coating over the surface. In some cases, the monomers, oligomers, polymers (e.g., low molecular weight polymers), or combinations thereof cross-link as the coating is formed while the solvent is removed from the surface. The resulting protective coating can then serve as a barrier to water loss from and/or oxidation of the produce or agricultural product, can protect the produce or agricultural product from biotic and abiotic stressors, can reduce or modify the respiration rate of the produce or agricultural product, and/or can otherwise reduce or modify the ripening rate of the produce or agricultural product.

Properties of the coating, such as thickness, cross-link density of monomers/oligomers/polymers, and permeability, can be varied to be suitable for a particular agricultural product by adjusting the specific composition of the coating agent, the specific composition of the solvent, the concentration of the coating agent in the solvent, and conditions of the coating deposition process (e.g., the amount of time the solution is applied to the surface of the produce or agricultural product before the solvent is removed, the temperature during the deposition process, the standoff distance between the spray head and the sample, and the spray angle). For example, too short an application time can result in incomplete coverage of the produce or agricultural product, whereas too long an application time can result in the produce or agricultural product potentially being damaged by the solvent. Accordingly, the solution can be applied to the surface of the produce or agricultural product for between 1 and 3,600 seconds, for example between 100 and 3,000 seconds or between 500 and 2,000 seconds. Furthermore, the concentration of the coating agent in the solvent can, for example, be in a range of 0.1 to 200 mg/mL or about 0.1 to 200 mg/mL, such as in a range of about 1 to 100 mg/mL, 1 to 50 mg/mL, 5 to 100 mg/mL, or 5 to 50 mg/mL.

The protective coatings formed from coating agents described herein can be edible coatings. The protective coatings can be substantially undetectable to the human eye, and can be odorless and/or tasteless. The protective coatings can have an average thickness in the range of about 0.1 microns to 300 microns, for example in the range of about 0.5 microns to 100 microns, 1 micron to 50 microns, 0.1 microns to 10 microns, or less than 10 microns. In some implementations, the protective coatings are entirely organic (e.g., organic in the agricultural sense rather than the chemistry sense). In some embodiments, the produce is a thin-skinned fruit or vegetable. For instance, the produce can be a berry or peach. In some embodiments, the produce can include a cut fruit surface (e.g., a cut apple surface).

Referring now to step 304 of process 300 (FIG. 3), after the agricultural products are coated to reduce their ripening rates, respiration rates, and/or mass loss rates, the coated products can be stored. During storage, the coated products ripen at a reduced rate as compared to similar products that have not been coated, thereby allowing the agricultural products to be stored for extended periods of time without ripening and subsequently spoiling.

Referring now to step 306 of process 300 (FIG. 3), as the desired date for ripening of the agricultural products approaches, the coating is removed (or at least partially removed) from the products in order to increase the ripening rate of the products. Removal of the coatings can, for example, be accomplished by soaking the agricultural products in a solvent in which the coatings are soluble or at least somewhat soluble. The solvent and soak time are preferably selected so as not to damage or otherwise degrade the quality of the agricultural products. The solvent can, for example, be water, ethanol, methanol, acetone, isopropanol, ethyl acetate, or combinations thereof. In order to minimize damage or degradation to the agricultural products, as well as to minimize processing times, it is preferable that the soak time for removing the coating be as low as possible, for example less than 10 minutes, less than 5 minutes, less than 3 minutes, less than 2 minutes, less than 1 minute, less than 50 seconds, less than 40 seconds, less than 30 seconds, less than 20 seconds, or less than 10 seconds.

In some embodiments, the solvent used to remove the coatings is heated, for example to at least 30° C., at least 40° C., or at least 50° C. Heating the solvent can increase the solubility of the coatings in the solvent, thereby allowing for lower application times to remove (or at least partially remove) the coatings and thereby increase the ripening rate of the agricultural products. Alternatively, the solvent used to remove the coating can be cooled, for example to less than 25° C., less than 20° C., less than 15° C., less than 14° C., less than 12° C., less than 10° C., less than 8° C., less than 6° C., less than 5° C., or less than 4° C. While reducing the temperature of the solvent can reduce the solubility of the coatings in the solvent, application of the solvent to the agricultural products can occur during sorting or processing steps for which it is preferable to maintain a reduced temperature. For example, in some embodiments the coating is removed along a packing line of the produce, for which the temperature may be typically kept at about 13° C. or less. In other embodiments, the coating is removed in a cold room in which the produce is stored, for which the temperature may be typically kept at about 4° C. or less. In each of these cases, it can be preferable that the solvent be at about ambient temperature in order to prevent breaking of the cold chain.

Coatings can alternatively be removed by methods other than soaking in a solvent. For example, the coatings can be mechanically removed, for example by placing the coated products on a brush bed. Or, the coated product can first be soaked in a solvent to weaken the coating, followed by mechanical removal of the coating. Furthermore, in some embodiments the coating is removed by storing the coated products in a moist environment (e.g., at a high relative humidity) and allowing the coating to be dissolved into the surrounding liquid.

As previously described, the protective coatings formed from coating agents described herein can reduce the ripening rate of the coated products, and removal of the protective coatings can subsequently increase the ripening rate of the products. For example, the coatings can be formulated to inhibit oxidation of the underlying produce, and can also be formulated to serve as a barrier to moisture, thereby reducing the rate of mass loss of the coated products. The coatings can also be formulated to reduce the respiration rate of the coated products. The inventors of the present disclosure have investigated correlations between ripening rates and changes in physical appearance of the outer portion of the produce, between ripening rates and mass loss rates of the produce, and between ripening rates and respiration rates of the produce. As further described below, ripening of produce can be much more highly correlated to respiration rates and in some cases mass loss rates than to changes in physical appearance. While in many cases produce ripens without detectable changes to its outer appearance, mass loss and shrinkage can be directly correlated to softening, which is a sign of ripening, and respiration can be directly correlated to physiological decay.

As used herein, the “respiration rate” of a product such as produce refers to the rate at which the product releases CO₂, and more specifically is the volume of CO₂ (at standard temperature and pressure) released per unit time per unit mass of the product. The respiration rate of produce can be measured by placing the produce in a closed container of known volume that is equipped with a CO₂ sensor, recording the CO₂ concentration within the container as a function of time, and then calculating the rate of CO₂ release required to obtain the measured concentration values.

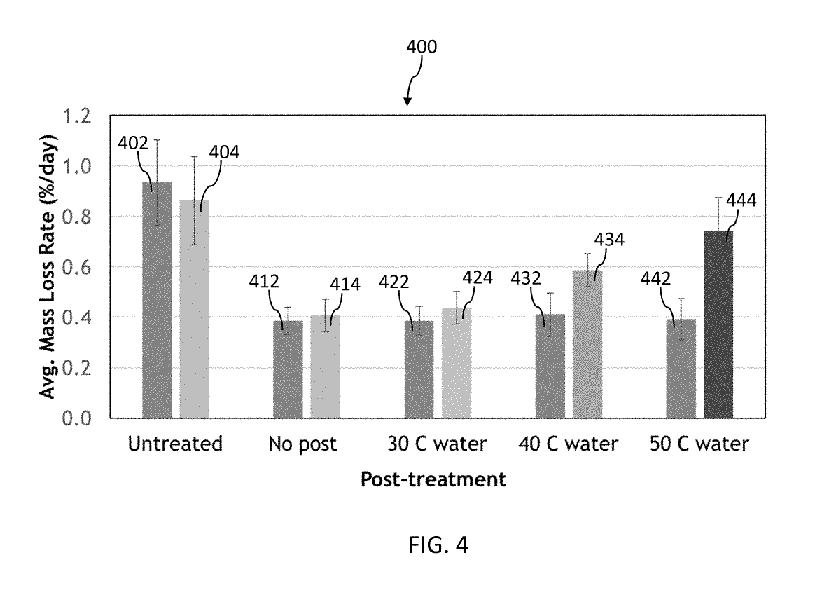

FIGS. 4-8 show the effects of forming coatings on avocados to reduce mass loss rates (and associated ripening rates), storing the coated avocados, and then removing the coatings to subsequently increase the mass loss rates (and associated ripening rates). Coatings on all avocados were formed as follows. First, a solid coating composition was formulated which included a mixture of 2-monoacylglyceride compounds and an additive. For the plots in FIGS. 4-8, the 2-monoacylglyceride compounds were 1,3-dihydroxypropan-2-yl palmitate (i.e., PA-2G), the additive was 2,3-dihydroxypropyl palmitate (i.e., PA-1G), and a mass ratio of the PA-2G to PA-1G in the coating composition was 70:30. However, coating compositions with similar properties can also be formed with other combinations of 1-monoacylglyceride compounds, 2-monoacylglyceride compounds, and/or other additives, as well as different mass ratios of PA-2G to PA-1G.

The coating composition was dissolved in a solvent that was 80% ethanol and 20% water by volume to form a solution. The concentration of the coating composition in the solvent was 10 mg/mL. The avocados were then dipped in the solution for about 30 seconds and agitated to ensure application of the solution over the entire surface, where on average each avocado had about 1 mL of the solution applied to its surface. The avocados were then removed from the solution and placed on drying racks, and the solvent was allowed to evaporate, resulting in the protective coatings being formed over the outer surfaces of each of the avocados.

Removal of all coatings was carried out by soaking the avocados in a solvent bath (e.g., water or ethanol), as indicated in the various figures and their accompanying descriptions. In each case, the volume of the solvent in the bath was equal to about 700 mL times the number of avocados soaked in the bath.

FIG. 4 is a plot 400 of mass loss rates for a batch of avocados that were coated and then subsequently had their coatings removed by soaking in water for 5 minutes. The avocados were all picked at about the same time and at about the same stage or ripeness. All coatings were formed at the same time, about 2-3 days after the avocados were picked, and subsequently removed at the same time, 2 days after the coatings were formed. During the entire study, all avocados were stored under ambient conditions (approximately 20° C. average temperature and about 40%-60% relative humidity). A first control group (corresponding to 402 and 404) was not coated or subjected to a subsequent soaking in water. A second control group (corresponding to 412 and 414) was coated but did not subsequently have the coating removed. A first test group (corresponding to 422 and 424) was coated and then subsequently soaked in 30° C. water for 5 minutes to remove the coating. A second test group (corresponding to 432 and 434) was coated and then subsequently soaked in 40° C. water for 5 minutes to remove the coating. A third test group (corresponding to 442 and 444) was coated and then subsequently soaked in 50° C. water for 5 minutes to remove the coating. Bars 402, 412, 422, 432, and 442 are average daily percent mass loss rates of the 5 groups of avocados measured during the time that the avocados had coatings thereon (apart from the first control group, which was uncoated). Bars 412, 422, 432, and 442 represent 4 distinct groups of avocados, each having coatings formed thereon at the same time, and mass loss rates were measured simultaneously for each of these groups while the avocados were coated with the coatings described herein. Bars 404, 414, 424, 434, and 444 are average daily percent mass loss rates of the 5 groups of avocados measured after the coatings were removed from the three test groups (but remained on the second control group).

As seen in FIG. 4, during the time that the avocados in the second control group and in the three test groups were coated, the untreated avocados (bar 402) experienced an average mass loss rate of 0.93% per day (i.e., greater than 0.9% per day), whereas the coated avocados (bars 412, 422, 432, and 442) experienced respective mass loss rates of 0.38% per day, 0.38% per day, 0.41% per day, and 0.39% per day, indicating that the coatings had reduced the mass loss rate by greater than a factor of 2. After the coatings were removed from the three test groups, the mass loss rates of the two control groups remained about the same. That is, the mass loss rate of the uncoated avocados (bar 404) was 0.86% per day, and the mass loss rate of the still coated avocados (bar 414) was 0.40% per day. However, the avocados soaked in 30° C. water experienced a small increase in mass loss rate, to 0.45% per day (bar 424), indicating, without wishing to be bound by theory, that the coatings had been partially removed and/or weakened and the ripening rate slightly increased. The avocados soaked in 40° C. water experienced a larger increase in mass loss rate, to 0.59% per day (bar 434), indicating, without wishing to be bound by theory, a more substantial removal of the coatings and a larger increase in ripening rate. The avocados soaked in 50° C. water experienced an even larger increase in mass loss rate, to 0.74% per day (bar 444), indicating, without wishing to be bound by theory, a more substantial removal of the coatings and an even larger increase in ripening rate. For the avocados soaked in 50° C. water, the mass loss rate after removal of the coating was more than 50% greater than the mass loss rate while the avocados were coated. In fact, for avocados soaked in 50° C. water, the mass loss rate was over 75% of the mass loss rate of the uncoated avocados during the same time period.

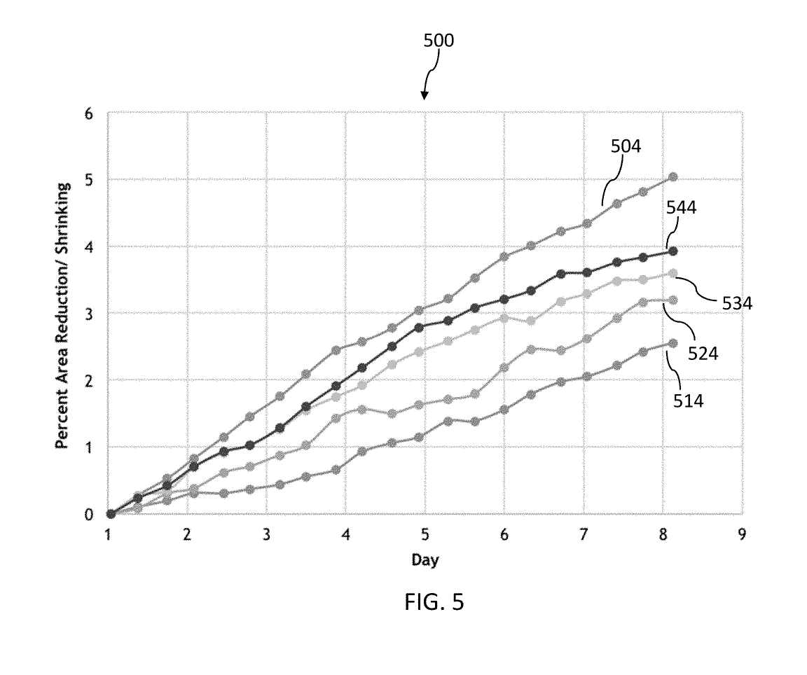

FIG. 5 shows plots 500 of the percent area reduction as a function of time for the avocados measured in FIG. 4 during the eight days after the coatings were removed from the three test groups. As seen in FIG. 5, after eight days, the cross sectional areas of the uncoated avocados (504) had decreased by 5.0%, whereas the cross sectional areas of the avocados that still had coatings thereon (514) had only decreased by 2.5%. The cross sectional areas of the avocados that had been soaked in 30° C. water (524) decreased by 3.2% after eight days. The cross sectional areas of the avocados that had been soaked in 40° C. water (534) decreased by 3.7% after eight days. The cross sectional areas of the avocados that had been soaked in 50° C. water (544) decreased by 3.9% after eight days.

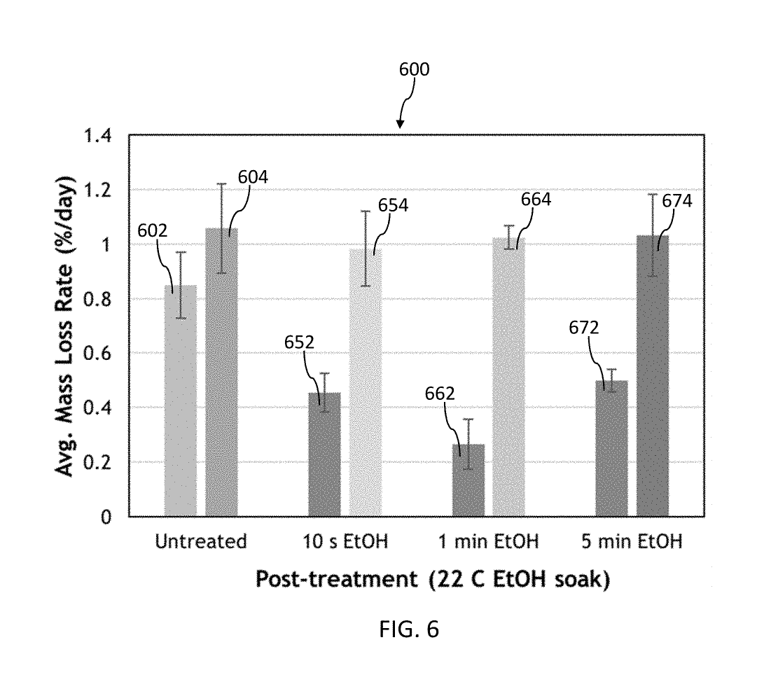

FIG. 6 is a plot 600 of mass loss rates for a batch of avocados that were coated and then subsequently had their coatings removed by soaking in substantially pure ethanol at 22° C. for different amounts of time. As with FIG. 4, the avocados were all picked at about the same time and at about the same stage of ripeness. All coatings were formed at the same time, about 2-3 days after the avocados were picked, and subsequently removed at the same time, 2 days after the coatings were formed. During the entire study, all avocados were stored under ambient conditions (approximately 20° C. average temperature and about 40%-60% relative humidity). A first control group (corresponding to 602 and 604) was not coated or subjected to a subsequent soaking in ethanol. A first test group (corresponding to 652 and 654) was coated and then subsequently soaked in 22° C. ethanol for 10 seconds to remove the coating. A second test group (corresponding to 662 and 664) was coated and then subsequently soaked in 22° C. ethanol for 1 minute to remove the coating. A third test group (corresponding to 672 and 674) was coated and then subsequently soaked in 22° C. ethanol for 5 minutes to remove the coating. Bars 602, 652, 662, and 672 are average daily percent mass loss rates of the 4 groups of avocados measured during the time that the avocados had coatings thereon (apart from the control group, which was uncoated). Bars 652, 662, and 672 represent 3 distinct groups of avocados, each having coatings formed thereon at the same time, and mass loss rates were measured simultaneously for each of these groups while the avocados were coated with coatings described herein. Bars 604, 654, 664, and 674 are average daily percent mass loss rates of the 4 groups of avocados measured after the coatings were removed from the three test groups.

For the avocados measured in FIG. 6, the mass loss rates of the avocados having coatings thereon (bars 652, 662, and 672) were substantially lower than those of the uncoated avocados (bar 602) measured at the same time. However, for all three soak times, the mass loss rate after soaking in ethanol (bars 654, 664, and 674) was about the same as the mass loss rate of the uncoated avocados (bar 604), indicating, without wishing to be bound by theory, that the ethanol soak substantially eliminated the effects of the coating for all three soak times. Specifically, the effects of the coating were substantially eliminated by soaking the avocados in ethanol for about 5 minutes or less, about 3 minutes or less, about 1 minute or less, less than 30 seconds, less than 20 seconds, and less than 12 seconds.

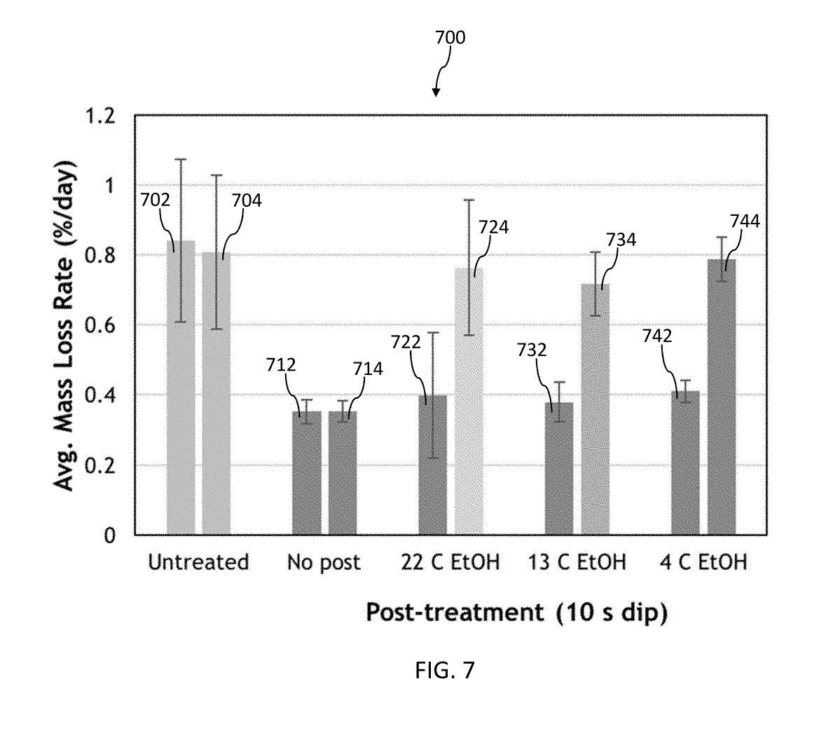

FIG. 7 is a plot 700 of mass loss rates for a batch of avocados that were coated and then subsequently had their coatings removed by soaking in substantially pure ethanol for 10 seconds at varying temperatures. The avocados were all picked at about the same time and at about the same stage or ripeness. All coatings were formed at the same time, about 2-3 days after the avocados were picked, and subsequently removed at the same time, 2 days after the coatings were formed. During the entire study, all avocados were stored under ambient conditions (approximately 20° C. average temperature and about 40%-60% relative humidity). A first control group (corresponding to 702 and 704) was not coated or subjected to a subsequent soaking in ethanol. A second control group (corresponding to 712 and 714) was coated but did not subsequently have the coating removed. A first test group (corresponding to 722 and 724) was coated and then subsequently soaked in 22° C. ethanol for 10 seconds to remove the coating. A second test group (corresponding to 732 and 734) was coated and then subsequently soaked in 13° C. ethanol for 10 seconds to remove the coating. A third test group (corresponding to 742 and 744) was coated and then subsequently soaked in 4° C. ethanol for 10 seconds to remove the coating. Bars 702, 712, 722, 732, and 742 are average daily percent mass loss rates of the 5 groups of avocados measured during the time that the avocados had coatings thereon (apart from the control group, which was uncoated). Bars 704, 714, 724, 734, and 744 are average daily percent mass loss rates of the 5 groups of avocados measured after the coatings were removed from the three test groups (but remained on the second control group).

For the avocados measured in FIG. 7, the mass loss rates of the avocados having coatings thereon (bars 712, 722, 732, and 742) were substantially lower than those of the uncoated avocados (bar 702) measured at the same time. Additionally, for all three soak temperatures, the mass loss rate after soaking in ethanol (bars 724, 734, and 744) was substantially similar to the mass loss rate of the uncoated avocados (bar 704), indicating, without wishing to be bound by theory, that even at reduced temperature and very short soak times, the ethanol soak substantially eliminated the effects of the coatings. Specifically, without wishing to be bound by theory, the effects of the coating were substantially eliminated by soaking the avocados in ethanol for less than 30 seconds at temperatures of about 22° C. or less, about 20° C. or less, about 17° C. or less, about 13° C. or less, about 10° C. or less, about 8° C. or less, or about 5° C. or less.

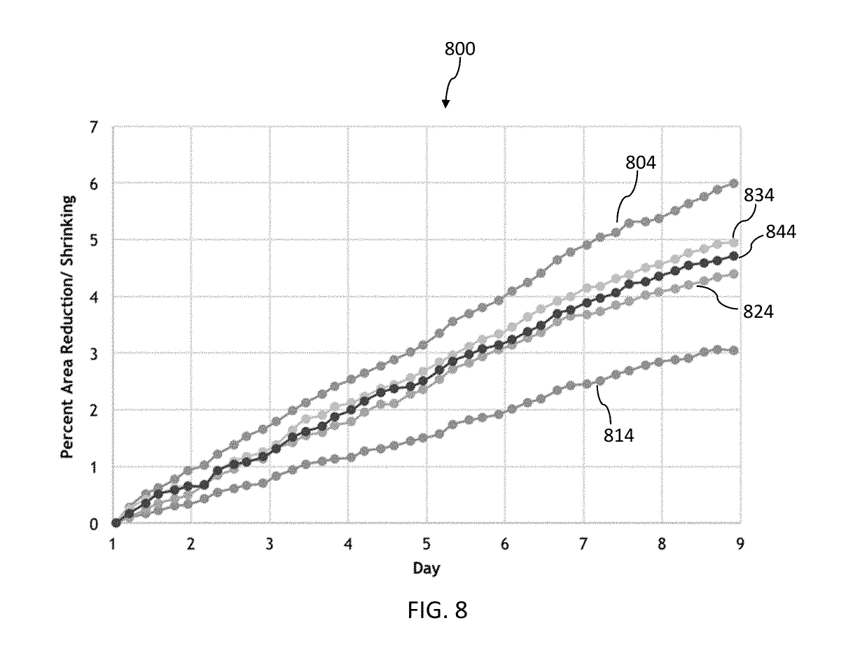

FIG. 8 shows plots 800 of the percent area reduction as a function of time for the avocados measured in FIG. 7 during the nine days after the coatings were removed from the three test groups. As seen in FIG. 8, after nine days, the cross sectional areas of the uncoated avocados (804) had decreased by about 6%, whereas the cross sectional areas of the avocados that still had coatings thereon (814) had only decreased by about 3%. The cross sectional areas of the avocados that had been soaked in 4° C. ethanol (844) decreased by 4.7% after nine days. The cross sectional areas of the avocados that had been soaked in 13° C. ethanol (834) decreased by 4.9% after nine days. The cross sectional areas of the avocados that had been soaked in 22° C. ethanol (824) decreased by 4.4% after nine days.

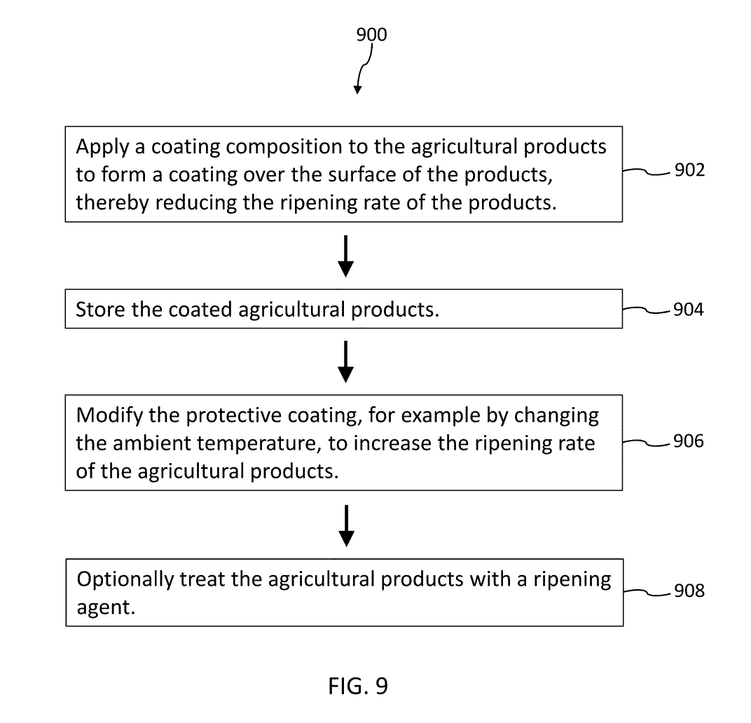

Another method 900 of controlling or delaying the rate of ripening in harvested produce and/or other agricultural products is illustrated in FIG. 9. The method 900 is similar to method 300 in FIG. 3, except that rather than removing the protective coating after storing the agricultural products, the coating is modified to alter its properties (e.g., to reduce its ability to protect the product from moisture loss and/or adsorption of oxygen or ethylene). For example, as detailed below, the coating can be formulated to serve as an effective barrier to moisture loss and/or adsorption of oxygen or ethylene while maintained within a first temperature range, but changing the ambient temperature of the produce to a temperature different from (e.g., higher than) the first temperature range can reduce the efficacy of the coating. Accordingly, during a first time period the coated produce can be stored at a first temperature, and the protective coating can subsequently be modified by heating the produce to a second temperature greater than the first temperature. As an example, the coating can be formed and the agricultural product stored all while the agricultural product is in a cold room (e.g., typically maintained at a temperature in a range of about 2° C. to about 15° C.), and the coating can be formulated to serve as an effective barrier while maintained at these colder temperatures but not at warmer temperatures (e.g., temperatures greater than about 15° C., for example typical room temperature, which is about 20° C.). As such, the ripening rate of the agricultural product can decrease while the product is in cold storage (as compared to similar agricultural products that are uncoated), but once the agricultural product is removed from cold storage and placed in ambient conditions (e.g., at an ambient temperature greater than about 15° C.), the ripening rate can be substantially similar to that of uncoated products that are at the same stage of ripening.

Referring to FIG. 9, first a coating composition is applied to the agricultural products to form a coating over a surface of the products (step 902). As with method 300, the coating can be applied either before or after harvesting of the agricultural product. The coating composition can, for example, include a plurality of monomers, oligomers, low molecular weight polymers, or combinations thereof (including functionalized monomers, oligomers, or low molecular weight polymers). In some embodiments, the coating composition is dissolved in a solvent to form a solution, the solution is applied to the agricultural products, and the solvent is then allowed to at least partially evaporate, thereby resulting in a coating comprising the constituents of the coating composition being formed over the surface of the products. In some embodiments, the coating composition can be formulated such that the coating serves as an effective barrier to moisture loss and/or adsorption of oxygen, ethylene, or other ripening agents while maintained within a first temperature range, and the agricultural products can be maintained at an ambient temperature that is within the first temperature range while being coated.

Next, the coated agricultural products are stored (step 904), for example during sorting, processing, and/or shipping. The agricultural products can be maintained at a first ambient temperature that is within the first temperature range during storage, thereby causing the agricultural products to ripen and optionally lose mass at a reduced rate during storage, and also delaying ripening and prolonging the life of the harvested products. In some embodiments, the agricultural products are coated and stored at substantially the same temperature. For example, the products can be both coated and stored in a cold room, in which the ambient temperature is controlled to be substantially constant throughout at all times.

Finally, when full ripening of the agricultural products is desired, the coating is modified (e.g., the properties of the coating are modified), such that efficacy of the coating is reduced, and correspondingly the respiration rate and/or ripening rate of the agricultural product increases (step 906). For example, products that are coated and stored in cold storage at an ambient temperature that is within the above referenced first temperature range can be removed from cold storage and placed in ambient conditions (e.g., at an ambient temperature that is not within the first temperature range). In some embodiments, changing the ambient temperature of the coated produce to a value that is not within the first temperature range causes the structure of the coating to be modified (e.g., to at least partially undergo a phase change), thereby reducing the efficacy of the coating and optionally causing the coated products to ripen at a rate substantially similar to that of similar uncoated agricultural products that are at the same stage of ripening.

As also shown in FIG. 9, after modifying the protective coating, the agricultural products may optionally be treated with a ripening agent in order to further accelerate ripening (step 908). For example, the products can be subjected to ethylene gas to accelerate ripening.

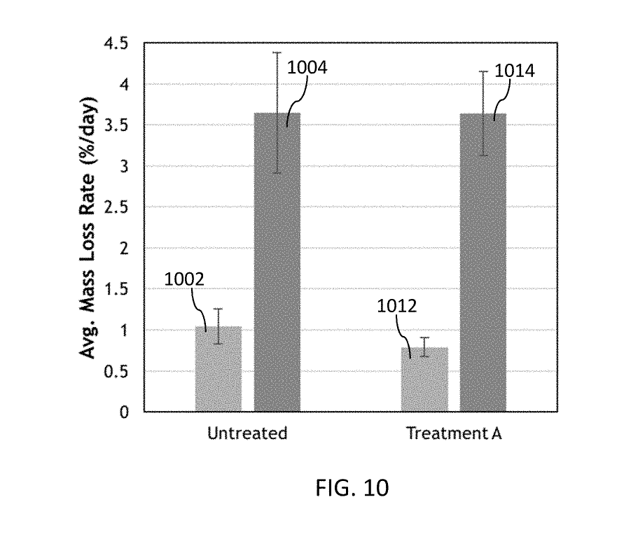

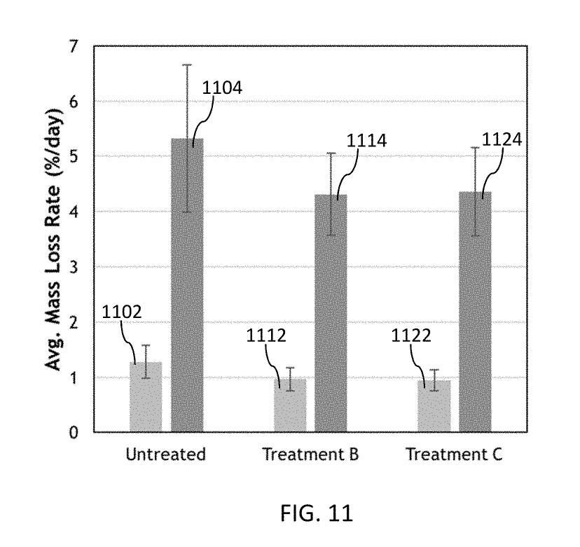

Coatings for which the properties and barrier efficacy exhibited the temperature dependence described above were formed over finger limes, with results shown in FIGS. 10 and 11. Coatings on all finger limes were formed as follows. First, a solid coating composition was formulated which included a mixture of 1,3-dihydroxypropan-2-yl palmitate (i.e., PA-2G) compounds and an additive. For the plot of FIG. 10 the additive was 1,3-dihydroxypropan-2-yl (9Z)-Octadecenoate (i.e., 2-glycero oleate or OA-2G), and for the plot of FIG. 11 the additive was (9Z)-Octadecenoic acid (i.e., oleic acid or OA). The coating composition was dissolved in a solvent that was substantially pure ethanol to form a solution. The concentration of the coating composition in the solvent was 10 mg/mL. The finger limes were then dipped in the solution for about 30 seconds and agitated to ensure application of the solution over the entire surface, where on average each finger lime had about 0.2 mL of the solution applied to its surface. The finger limes were then removed from the solution and placed on drying racks, and the solvent was allowed to evaporate, resulting in the protective coatings being formed over the outer surfaces of each of the finger limes.

FIG. 10 shows plots of the average mass loss rate for untreated (i.e., uncoated) finger limes at 4.4° C. (bar 1002) and at 20° C. (bar 1004), as well as average mass loss rate for coated finger limes at 4.4° C. (bar 1012) and at 20° C. (bar 1014). For the coated finger limes (bars 1012 and 1014), the mass ratio of OA-2G to PA-2G in the coating composition was 25:75. For the measurements of both of these sets of finger limes, the limes were first held at 20° C. for 1 to 1.5 days while mass loss rates were measured, and were then cooled to and held at 4.4° C. for 2 to 2.5 days while mass loss rates were measured. At 20° C., the average mass loss rates for both the uncoated (1004) and coated (1014) finger limes were about the same (both were about 3.6% per day). However, after cooling to 4.4° C., the uncoated finger limes (1002) experienced an average mass loss rate greater than 1% per day, whereas the coated finger limes (1012) experienced an average mass loss rate less than 0.8% per day. Thus the average mass loss rate of uncoated finger limes (1002) was more than 25% greater than that of the coated finger limes (1012) at 4.4° C. Without wishing to be bound by theory, these results indicate that the coatings were more effective at reducing mass loss rates of the finger limes at 4.4° C., than at higher temperatures such as 20° C.

FIG. 11 also shows plots of the average mass loss rate for untreated (i.e., uncoated) finger limes at 4.4° C. (bar 1102) and at 20° C. (bar 1104), as well as average mass loss rate for 2 groups of coated finger limes coated with different coating compositions than those used in FIG. 10. For the first group of coated finger limes (bars 1112 and 1114), the mass ratio of OA to PA-2G in the coating composition was 30:70. Bar 1112 corresponds to the average mass loss rate at 4.4° C., and bar 1114 corresponds to the average mass loss rate at 20° C. For the second group of coated finger limes (bars 1122 and 1124), the mass ratio of OA to PA-2G in the coating composition was 40:60. Bar 1122 corresponds to the average mass loss rate at 4.4° C., and bar 1124 corresponds to the average mass loss rate at 20° C. For the measurements of all three sets of finger limes in FIG. 11, the limes were first held at 20° C. for 1 to 1.5 days while mass loss rates were measured, and were then cooled to and held at 4.4° C. for 2 to 2.5 days while mass loss rates were measured. At 20° C., the uncoated finger limes (1104) experienced an average mass loss rate of 5.32% per day, whereas the coated finger limes in the first group (1114) experienced an average mass loss rate of 4.31% per day, and the coated finger limes in the second group (1124) experienced an average mass loss rate of 4.35% per day. Thus, at 20° C., the average mass loss rate of uncoated finger limes (1104) was about 23% greater than that of the coated finger limes (1114) in the first group and about 22% greater than that of the coated finger limes (1124) in the second group. After cooling to 4.4° C., the uncoated finger limes (1102) experienced an average mass loss rate of 1.27% per day, whereas the coated finger limes in the first group (1112) experienced an average mass loss rate of 0.98% per day, and the coated finger limes in the second group (1122) experienced an average mass loss rate of 0.95% per day. Thus, at 4.4° C., the average mass loss rate of uncoated finger limes (1102) was about 30% greater than that of the coated finger limes (1112) in the first group and about 34% greater than that of the coated finger limes (1122) in the second group. Hence, without wishing to be bound by theory, the efficacy of the coating (as determined by percent difference in mass loss rate of coated finger limes as compared to uncoated finger limes) was greater at 4.4° C. than at 20° C.

While the finger limes in FIGS. 10 and 11 were first held at 20° C. and then cooled to 4.4° C., the sequence can be reversed as well. For example, the finger limes of FIG. 10 can first be held at 4.4° C. and then heated to 20° C. In this case, the coating can be effective at reducing the respiration rate and/or delaying ripening at the initial temperature (4.4° C.) but less effective at the final temperature (20° C.). Similarly, the finger limes of FIG. 11 can first be held at 4.4° C. and then heated to 20° C., in which case the efficacy of the coating (as determined by percent difference in mass loss rate of coated finger limes as compared to uncoated finger limes) can be greater at the initial temperature (4.4° C.) than at the final temperature (20° C.).

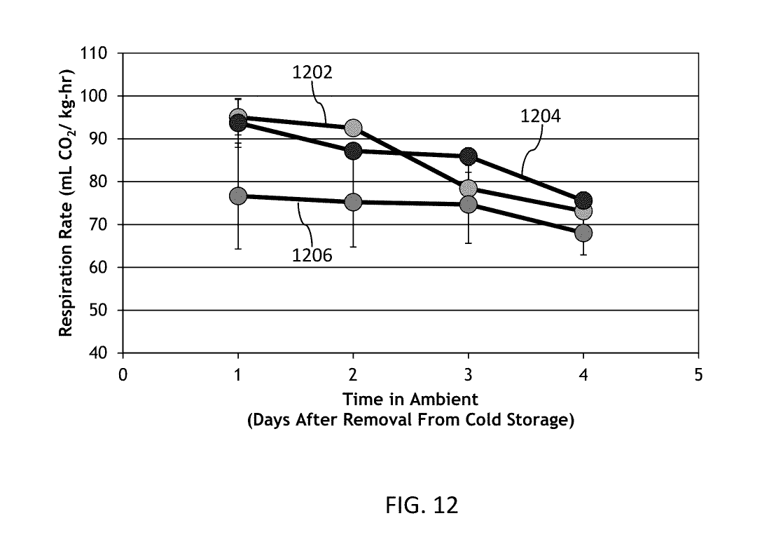

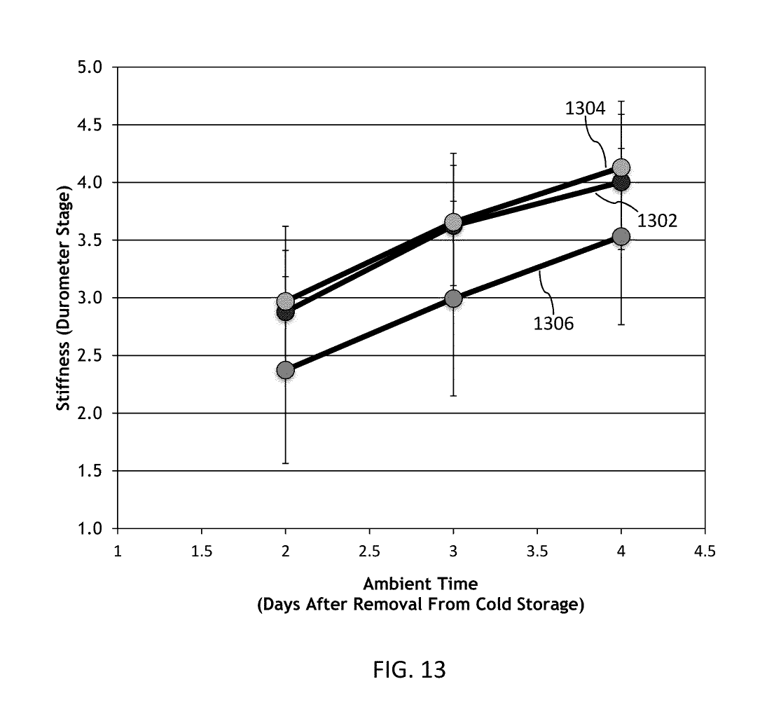

FIGS. 12 and 13 show plots of respiration rate and stiffness (durometer stage), respectively, of avocados as a function of time removed from cold storage. The avocados were stored at 5° C. and 85% relative humidity for 5 weeks and then removed from cold storage and maintained in ambient conditions while the measurements corresponding to FIGS. 12 and 13 were made. Plots 1202 and 1302 correspond to a group of 90 untreated avocados, plots 1206 and 1306 correspond to a group of 90 avocados that were coated prior to being placed in cold storage, and plots 1204 and 1304 correspond to a group of 90 avocados that were coated prior to being placed in cold storage but had their coatings removed immediately upon removal from cold storage. Coating composition and deposition and removal procedures are described in detail in Example 5 below.

As seen in FIG. 12, 1 day after removal from cold storage and removal of the coatings from the avocados corresponding to plot 1204, the respiration rates of the untreated avocados (1202) and of the avocados that had their coatings removed (1204) were substantially greater (more than 20% greater) than the respiration rates of the coated avocados. Over the next 3 days, the respiration rates of the coated avocados (1206) dropped slightly, while the respiration rates of the untreated avocados (1202) and of the avocados that had their coatings removed (1204) dropped more significantly, such that 4 days after removal from cold storage the respiration rates of the avocados corresponding to plots 1202 and 1204 were only slightly greater (about 10% greater) than those of the coated avocados (1206).

Without wishing to be bound by theory, it is believed that the coating serves to both reduce the respiration rate of the avocados, thereby delaying the ripening process, and also to lower and widen the climacteric respiration peak of the avocados. The respiration measurements in FIG. 12 are consistent with this phenomenon. One day after removal from cold storage, the untreated avocados and avocados with coatings removed had essentially the same high respiration rate, whereas the avocados with coatings intact had a lower respiration rate. The respiration rate of all avocados then decreased over time, since the climacteric respiration peak had already occurred. The rate of decrease for the respiration rates of the untreated avocados and avocados with coatings removed was very similar. The rate of decrease of respiration for the avocados with coatings intact was lower than that of the untreated avocados and of the avocados with the coatings removed.

FIG. 13 shows plots of stiffness (durometer stage) as a function of time removed from cold storage for the same avocados measured in FIG. 12. Stiffness was measured with a standard durometer, where increasing durometer stage corresponds to a lower stiffness. A durometer stage of 4.0 for avocados is typically considered sufficiently ripe for consumption. As shown, between days 2 and 4 after removal from cold storage, the durometer stage reading for all 3 groups of avocados increased, indicating that the avocados were becoming less stiff (and therefore more ripe). However, the avocados having their coatings intact (1306) were stiffer than the untreated avocados (1302) and the avocados for which the coatings had been removed (1304). Without wishing to be bound by theory, these results indicate that the coated avocados ripened at a slower rate, whereas removing the coating caused avocados to ripen at a rate similar to that of untreated avocados.

In one or more embodiments, the coatings described herein comprise cross-linked monomers, oligomers, low molecular weight polymers, or combinations thereof. In some embodiments, the monomers, oligomers, low molecular weight polymers, or combinations thereof crosslink on the surface of the produce.

In some embodiments, the harvested produce is stored for at least 1 day with the coating thereon prior to removal of the coating. In some embodiments, the produce is coated before the produce is harvested, and the coating is at least partially removed after the produce is harvested. In some embodiments, the step of at least partially removing the coating comprises rinsing the produce in a solvent. For instance, the solvent can be heated to at least 30° C., at least 40° C., or at least 50° C. The solvent can comprise water, ethanol, or combinations thereof. In some embodiments, the solvent is ethanol and the ethanol is cooled to 13° C. or below.

In some embodiments, the coating reduces a rate of respiration of the produce. Reducing the rate of respiration can include reducing the rate of moisture loss from the produce and/or reducing the rate of adsorption of gases or vapors such as oxygen or ethylene. In some embodiments, the coating causes the rate of respiration of the produce to decrease, and the at least partially removing of the coating causes the rate of respiration of the produce to increase. In some embodiments, the coating is substantially undetectable to the human eye when applied to the produce. In some embodiments, the coating is substantially odorless or tasteless when applied to the produce. In some embodiments, the coating is formulated to reduce water loss from the produce.

In some embodiments, the coating composition includes at least one of monomers, oligomers, and low molecular weight polymers. The monomers, oligomers, and/or low molecular weight polymers can be functionalized monomers, oligomers, and/or low molecular weight polymers. In some embodiments, the coating composition includes monoacylglycerides.

In some embodiments, the protective coating has a thickness less than about 1 micron or less than about 10 microns. In some embodiments, the coating has an average transmittance of at least 60% for light in the visible range.

In some embodiments, the present disclosure provides for treating the produce with a ripening agent after the at least partially removing the coating. For instance, treating the produce with a ripening agent can comprise gassing the produce with ethylene.

In any of the embodiments described herein, the coating can be applied either pre-harvest or post-harvest. That is, the coating can be applied while the produce is still attached to the plant from which it grows (e.g., a piece of fruit can still be on the vine). Alternatively, the coatings can be applied when the produce has been harvested (e.g., picked). The coating can be applied immediately after harvest or within hours or days of harvest. The coating can be applied, for instance, when the produce is packaged (e.g., in a shipping container).

In some embodiments, the coating of the produce, the storing and the produce, and the removal and/or modification of the coating are carried out by multiple parties. For example, a farmer could apply the coatings to produce and then transfer the produce to a shipper, distributor, or retailer, who can store the coated produce and then subsequently remove or otherwise modify the coatings. Alternatively, the farmer can apply the coatings to the produce also store the produce, after which the farmer can transfer the produce to a distributor or retailer who subsequently removes or otherwise modifies the coating.

In some cases where multiple parties are involved, the first party (e.g., the party that forms the coatings) may optionally provide instructions or recommendations about treating or handling the produce, either written or oral, indicating one or more of the following: (i) that the produce has a coating thereon, and that the coating is to be removed or modified prior to sale of the produce; and/or (ii) conditions and/or methods that are suitable for removing or modifying the coatings. While the instructions or recommendations can be supplied by the first party directly with the coated produce (e.g., on packaging in which produce is stored), the instructions or recommendations may alternatively be supplied separately, for example on a website owned or controlled by the first party, or in advertising or marketing material provided by or on behalf of the first party.

In view of the above, it is recognized that in some cases, a party that coats an agricultural product according to one or more methods described herein (i.e., a first party) may not directly store the product and/or remove or modify the coating, but can instead direct (e.g., can instruct or request) a second party to store the product and/or remove or modify the coating. That is, even if the first party does not store the agricultural product and/or remove or modify the coating, the first party may still cause the agricultural product to be stored and/or cause the coating to be removed or modified, for example by providing instructions or recommendations as described above. Similarly, while the party that removes or modifies the coating (i.e., the second party) may not form the coating over the agricultural product, the second party may still cause the agricultural product to be coated, for example by providing instructions or recommendations to the first party that the agricultural products be coated prior to the second party receiving the products. Accordingly, as used herein, the act of applying a plant extract composition to a product (e.g., a plant or agricultural product) also includes directing or instructing another party to apply the plant extract composition to the product, or causing the plant extract composition to be applied to the product. Additionally, as used herein, the act of removing the coating from a product (e.g., a plant or agricultural product) also includes directing or instructing another party to remove the coating from the product, or causing the coating to be removed from the product.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Examples 1-3 show the effects of forming coatings on avocados, storing the coated avocados, and then removing the coatings. Coatings on all of the coated avocados in Examples 1-3 were formed as follows. First, a solid coating composition was formulated which included a mixture of 1,3-dihydroxypropan-2-yl palmitate (i.e., PA-2G) and 2,3-dihydroxypropyl palmitate (i.e., PA-1G), and a mass ratio of the PA-2G to PA-1G in the coating composition was 70:30. The coating composition was dissolved in a solvent that was about 80% ethanol and 20% water by volume to form a solution. The concentration of the coating composition in the solvent was 10 mg/mL. The avocados were then dipped in the solution for about 30 seconds and agitated to ensure application of the solution over the entire surface, where on average each avocado had about 1 mL of the solution applied to its surface. The avocados were then removed from the solution and placed on drying racks, and the solvent was allowed to evaporate, resulting in the protective coatings being formed over the outer surfaces of each of the avocados.

In each of Examples 1-3, subsequent removal of all coatings was carried out by soaking the avocados in a solvent bath (e.g., water or ethanol), as indicated in the various figures and their accompanying descriptions. In each case, the volume of the solvent in the bath was equal to 700 mL times the number of avocados soaked in the bath.

### Example 1

### Coatings Formed on Avocados and Removed by Soaking in Water—Effect of Water Temperature

Fifty avocados were picked at the same time and at about the same stage or ripeness and divided into 5 groups containing 10 avocados each. All coatings were formed at the same time, about 2-3 days after the avocados were picked, and subsequently removed at the same time, 2 days after the coatings were formed. During the entire study, all avocados were stored under ambient conditions (approximately 20° C. average temperature and about 40%-60% relative humidity).

The results are shown in FIGS. 4 and 5. A first control group (corresponding to 402 and 404) was not coated or subjected to a subsequent soaking in water. A second control group (corresponding to 412 and 414) was coated but did not subsequently have the coating removed. A first test group (corresponding to 422 and 424) was coated and then subsequently soaked in 30° C. water for 5 minutes to remove the coating. A second test group (corresponding to 432 and 434) was coated and then subsequently soaked in 40° C. water for 5 minutes to remove the coating. A third test group (corresponding to 442 and 444) was coated and then subsequently soaked in 50° C. water for 5 minutes to remove the coating. Bars 402, 412, 422, 432, and 442 are average daily percent mass loss rates of the 5 groups of avocados measured during the time that the avocados had coatings thereon (apart from the first control group, which was uncoated). Bars 412, 422, 432, and 442 represent 4 distinct groups of avocados, each having coatings formed thereon at the same time, and mass loss rates were measured simultaneously for each of these groups while the avocados were coated with the coatings described herein. Bars 404, 414, 424, 434, and 444 are average daily percent mass loss rates of the 5 groups of avocados measured after the coatings were removed from the three test groups (but remained on the second control group).

As seen in FIG. 4, during the time that the avocados in the second control group and in the three test groups were coated, the untreated avocados (bar 402) experienced an average mass loss rate of 0.93% per day, whereas the coated avocados (bars 412, 422, 432, and 442) experienced respective mass loss rates of 0.38% per day, 0.38% per day, 0.41% per day, and 0.39% per day. After the coatings were removed from the three test groups, the mass loss rate of the uncoated avocados (bar 404) was 0.86% per day, and the mass loss rate of the still coated avocados (bar 414) was 0.40% per day. The avocados soaked in 30° C. water experienced a small increase in mass loss rate to 0.45% per day (bar 424). The avocados soaked in 40° C. water experienced a larger increase in mass loss rate, to 0.59% per day (bar 434). The avocados soaked in 50° C. water experienced an even larger increase in mass loss rate, to 0.74% per day (bar 444).

FIG. 5 shows plots 500 of the percent area reduction as a function of time for the avocados measured in FIG. 4 during the eight days after the coatings were removed from the three test groups. As seen in FIG. 5, after eight days, the cross sectional areas of the uncoated avocados (504) had decreased by 5.0%, the cross sectional areas of the avocados that still had coatings thereon (514) had decreased by 2.5%, the cross sectional areas of the avocados that had been soaked in 30° C. water (524) decreased by 3.2%, the cross sectional areas of the avocados that had been soaked in 40° C. water (534) decreased by 3.7%, and the cross sectional areas of the avocados that had been soaked in 50° C. water (544) decreased by 3.9%.

### Example 2

### Coatings Formed on Avocados and Removed by Soaking in Ethanol—Effect of Soak Time

Forty avocados were picked at the same time and at about the same stage of ripeness and divided into 4 groups of 10 avocados each. All coatings were formed at the same time, about 2-3 days after the avocados were picked, and subsequently removed at the same time, 2 days after the coatings were formed. During the entire study, all avocados were stored under ambient conditions (approximately 20° C. average temperature and about 40%-60% relative humidity).

The results are shown in FIG. 6. A first control group (corresponding to 602 and 604) was not coated or subjected to a subsequent soaking in ethanol. A first test group (corresponding to 652 and 654) was coated and then subsequently soaked in 22° C. ethanol for 10 seconds to remove the coating. A second test group (corresponding to 662 and 664) was coated and then subsequently soaked in 22° C. ethanol for 1 minute to remove the coating. A third test group (corresponding to 672 and 674) was coated and then subsequently soaked in 22° C. ethanol for 5 minutes to remove the coating. Bars 602, 652, 662, and 672 are average daily percent mass loss rates of the 4 groups of avocados measured during the time that the avocados had coatings thereon (apart from the control group, which was uncoated). Bars 652, 662, and 672 represent 3 distinct groups of avocados, each having coatings formed thereon at the same time, and mass loss rates were measured simultaneously for each of these groups while the avocados were coated with coatings described herein. Bars 604, 654, 664, and 674 are average daily percent mass loss rates of the 4 groups of avocados measured after the coatings were removed from the three test groups.

During the times that the three groups of avocados were coated (corresponding to bars 652, 662, and 672), the uncoated avocados (bar 602) exhibited an average mass loss rate of 0.85% per day, while the coated avocados (bars 652, 662, and 672) exhibited average mass loss rates of 0.45% per day, 0.26% per day, and 0.50% per day, respectively. After the coatings were removed from the coated avocados, the mass loss rate of the previously uncoated control group (bar 604) was 1.06% per day, the mass loss rate of the avocados soaked in 22° C. ethanol for 10 seconds (bar 654) was 0.98% per day, the mass loss rate of the avocados soaked in 22° C. ethanol for 1 minute (bar 664) was 1.02% per day, and the mass loss rate of the avocados soaked in 22° C. ethanol for 5 minutes (bar 674) was 1.03% per day.

### Example 3

### Coatings Formed on Avocados and Removed by Soaking in Ethanol—Effect of Soak Temperature

Fifty avocados were picked at the same time and at about the same stage or ripeness and divided into 5 groups of 10 avocados each. All coatings were formed at the same time, about 2-3 days after the avocados were picked, and subsequently removed at the same time, 2 days after the coatings were formed. During the entire study, all avocados were stored under ambient conditions (approximately 20° C. average temperature and about 40%-60% relative humidity).

The results are shown in FIGS. 7 and 8. A first control group (corresponding to 702 and 704) was not coated or subjected to a subsequent soaking in ethanol. A second control group (corresponding to 712 and 714) was coated but did not subsequently have the coating removed. A first test group (corresponding to 722 and 724) was coated and then subsequently soaked in 22° C. ethanol for 10 seconds to remove the coating. A second test group (corresponding to 732 and 734) was coated and then subsequently soaked in 13° C. ethanol for 10 seconds to remove the coating. A third test group (corresponding to 742 and 744) was coated and then subsequently soaked in 4° C. ethanol for 10 seconds to remove the coating. Bars 702, 712, 722, 732, and 742 are average daily percent mass loss rates of the 5 groups of avocados measured during the time that the avocados had coatings thereon (apart from the control group, which was uncoated). Bars 704, 714, 724, 734, and 744 are average daily percent mass loss rates of the 5 groups of avocados measured after the coatings were removed from the three test groups (but remained on the second control group). Each bar represents a distinct group of 10 avocados.

During the times that the three test groups of avocados (corresponding to bars 722, 732, and 742) were coated, the uncoated avocados (bar 702) exhibited an average mass loss rate of 0.84% per day, the coated avocados of the second control group (bar 712) exhibited an average mass loss rate of 0.35% per day, the coated avocados of the first test group (bar 722) exhibited an average mass loss rate of 0.40% per day, the coated avocados of the second test group (bar 732) exhibited an average mass loss rate of 0.38% per day, and the coated avocados of the third test group (bar 742) exhibited an average mass loss rate of 0.41% per day. After the coatings were removed from the avocados of the three test groups, the mass loss rate of the previously uncoated avocados of the first control group (bar 704) was 0.81% per day, the mass loss rate of the coated avocados of the second control group (bar 714) was 0.35% per day, the mass loss rate of the avocados soaked in 22° C. ethanol for 10 seconds (bar 724) was 0.76% per day, the mass loss rate of the avocados soaked in 13° C. ethanol for 10 seconds (bar 734) was 0.72% per day, and the mass loss rate of the avocados soaked in 4° C. ethanol for 10 seconds (bar 744) was 0.79% per day.

FIG. 8 shows plots 800 of the percent area reduction as a function of time for the avocados measured in FIG. 7 during the nine days after the coatings were removed from the three test groups. As seen in FIG. 8, after nine days, the cross sectional areas of the uncoated avocados of the first control group (804) had decreased by 6.0%, the cross sectional areas of the avocados of the second control group that still had coatings thereon (814) had decreased by 3.0%, the cross sectional areas of the avocados that had been soaked in 4° C. ethanol for 10 seconds (844) had decreased by 4.7%, the cross sectional areas of the avocados that had been soaked in 13° C. ethanol for 10 seconds (834) had decreased by 4.9%, and the cross sectional areas of the avocados that had been soaked in 22° C. ethanol for 10 seconds (824) had decreased by 4.4%.

### Example 4

### Coatings Formed on Finger Limes

Coatings formed over finger limes, for which results are shown in FIGS. 10 and 11, were formed as follows. First, three solid coating composition were formulated. The first coating composition was formed of 1,3-dihydroxypropan-2-yl palmitate (i.e., PA-2G) and 1,3-dihydroxypropan-2-yl (9Z)-Octadecenoate (i.e., 2-glycero oleate or OA-2G) mixed at a mass ratio of 3:1. The second coating composition was formed of PA-2G and (9Z)-Octadecenoic acid (i.e., oleic acid or OA) mixed at a mass ratio of 7:3. The third coating composition was formed of PA-2G and (9Z)-Octadecenoic acid (i.e., oleic acid or OA) mixed at a mass ratio of 3:2. The first coating composition was dissolved in substantially pure ethanol at a concentration of 10 mg/mL to form a first solution. The second coating composition was dissolved in substantially pure ethanol at a concentration of 10 mg/mL to form a second solution. The third coating composition was dissolved in substantially pure ethanol at a concentration of 10 mg/mL to form a third solution.

A first set of substantially similar finger limes (i.e., harvested at the same time, similar stage of ripening) were separated into two groups of 24 finger limes. The first group of finger limes was untreated, and the second group was dipped in the first solution for about 30 seconds and agitated to ensure application of the first solution over the entire surface of each finger lime, where on average each finger lime had about 0.2 mL of the first solution applied to its surface. The finger limes were then removed from the solution and placed on drying racks, and the solvent was allowed to evaporate, resulting in the protective coatings being formed over the outer surfaces of each of the finger limes of the second group.

FIG. 10 shows plots of the average mass loss rate for untreated (i.e., uncoated) finger limes of the first group at 4.4° C. (bar 1002) and at 20° C. (bar 1004), as well as average mass loss rates for coated finger limes of the second group (i.e., coated with PA-2G and OA-2G at a mass ratio of 3:1) at 4.4° C. (bar 1012) and at 20° C. (bar 1014). For the measurements of both of these groups of finger limes, the limes were first held at 20° C. for 1 to 1.5 days while mass loss rates were measured, and were then cooled to and held at 4.4° C. for 2 to 2.5 days while mass loss rates were measured. At 20° C., the average mass loss rate for both the uncoated (1004) and coated (1014) finger limes was 3.6% per day. After cooling to 4.4° C., the uncoated finger limes (1002) experienced an average mass loss rate of 1.0% per day, whereas the coated finger limes (1012) experienced an average mass loss rate of 0.8% per day.

A second set of substantially similar finger limes (i.e., harvested at the same time, similar stage of ripening) were separated into three groups of 24 finger limes. The first group of finger limes was untreated. The second group was dipped in the second solution (i.e., PA-2G and OA at a mass ratio of 7:3) for about 30 seconds and agitated to ensure application of the second solution over the entire surface of each finger lime, where on average each finger lime had about 0.2 mL of the second solution applied to its surface. The third group was dipped in the third solution (i.e., PA-2G and OA at a mass ratio of 3:2) for about 30 seconds and agitated to ensure application of the third solution over the entire surface of each finger lime, where on average each finger lime had about 0.2 mL of the third solution applied to its surface. The finger limes were then removed from their respective solutions and placed on drying racks, and the solvent was allowed to evaporate, resulting in the protective coatings being formed over the outer surfaces of each of the finger limes of the second and third groups.

FIG. 11 shows plots of the average mass loss rate for untreated (i.e., uncoated) finger limes of the first group at 4.4° C. (bar 1102) and at 20° C. (bar 1104), average mass loss rates for the coated finger limes of the second group (i.e., PA-2G and OA at a mass ratio of 7:3) at 4.4° C. (bar 1112) and at 20° C. (bar 1114), and average mass loss rates for the coated finger limes of the third group (i.e., PA-2G and OA at a mass ratio of 3:2) at 4.4° C. (bar 1122) and at 20° C. (bar 1124). For the measurements of each of these groups of finger limes, the limes were first held at 20° C. for 1 to 1.5 days while mass loss rates were measured, and were then cooled to and held at 4.4° C. for 2 to 2.5 days while mass loss rates were measured. At 20° C., the uncoated finger limes of the first group (1104) experienced an average mass loss rate of 5.32% per day, the coated finger limes of the second group (1114) experienced an average mass loss rate of 4.31% per day, and the coated finger limes of the third group (1124) experienced an average mass loss rate of 4.35% per day. After cooling to 4.4° C., the uncoated finger limes of the first group (1102) experienced an average mass loss rate of 1.27% per day, the coated finger limes of the second group (1112) experienced an average mass loss rate of 0.98% per day, and the coated finger limes of the third group (1122) experienced an average mass loss rate of 0.95% per day.

### Example 5

### Coatings Formed on Avocados and Removed by Soaking in Ethanol—Effect on Respiration Rate and Stiffness

Coatings formed over avocados, for which results are shown in FIGS. 12 and 13, were formed as follows. First, a solid coating composition was formed by combining 2,3-dihydroxypropan-2-yl (9Z)-Octadecenoate (i.e., 1-glycero oleate or OA-1G), 2,3-dihydroxypropan-2-yl palmitate (i.e., PA-1G), 1,3-dihydroxypropan-2-yl palmitate (i.e., PA-2G), and hexadecanoic acid (i.e., palmitic acid or PA) at mass ratios of 30:30:5:35. The solid coating composition was dissolved in substantially pure ethanol at a concentration of 7.5 mg/mL to form a solution.

A set of substantially similar avocados (i.e., harvested at the same time, similar stage of ripening) was divided into three groups, each group having 90 avocados. The first group of avocados was untreated, and the avocados of the second and third groups were coated with the solution by spraying the solution over the entire surface of each avocado and allowing the ethanol to evaporate. All three groups of avocados were then stored at 5° C. and 85% relative humidity for 5 weeks, after which all three groups were removed from cold storage and the avocados of the second group were each dipped in ethanol (at room temperature) for approximately 1 second to remove their coatings (the avocados of the third group were not dipped in ethanol and thus their coatings remained intact). The avocados were then maintained at ambient temperature and humidity for 4 days while respiration rates and stiffness (firmness) of the avocados were measured (FIGS. 12 and 13, respectively). Respiration rates were determined by storing the avocados in a closed container of known volume that was equipped with an infrared-based CO₂ sensor, recording the CO₂ concentration within the container as a function of time, and then calculating the rate of CO₂ release required to obtain the measured concentration values. Stiffness was measured with a standard durometer, where increasing durometer stage corresponds to a lower stiffness. A durometer stage of 4.0 for avocados is typically considered sufficiently ripe for consumption.

FIG. 12 shows plots of respiration rates as a function of time after removal from cold storage for the untreated avocados (1202), the avocados which had their coatings removed (1204), and the avocados which remained coated (1206). One day after removal from cold storage, the untreated avocados (1202) exhibited a respiration rate of 95.0 mL CO₂/kg-hr, the avocados which had their coatings removed (1204) exhibited a respiration rate of 93.7 mL CO₂/kg-hr, and the avocados which remained coated (1206) exhibited a respiration rate of 76.6 mL CO₂/kg-hr. Two days after removal from cold storage, the untreated avocados (1202) exhibited a respiration rate of 92.5 mL CO₂/kg-hr, the avocados which had their coatings removed (1204) exhibited a respiration rate of 87.2 mL CO₂/kg-hr, and the avocados which remained coated (1206) exhibited a respiration rate of 75.2 mL CO₂/kg-hr. Three days after removal from cold storage, the untreated avocados (1202) exhibited a respiration rate of 78.4 mL CO₂/kg-hr, the avocados which had their coatings removed (1204) exhibited a respiration rate of 85.9 mL CO₂/kg-hr, and the avocados which remained coated (1206) exhibited a respiration rate of 74.7 mL CO₂/kg-hr. Four days after removal from cold storage, the untreated avocados (1202) exhibited a respiration rate of 73.1 mL CO₂/kg-hr, the avocados which had their coatings removed (1204) exhibited a respiration rate of 75.6 mL CO₂/kg-hr, and the avocados which remained coated (1206) exhibited a respiration rate of 68.0 mL CO₂/kg-hr.

FIG. 13 shows plots of stiffness (durometer stage) as a function of time after removal from cold storage for the untreated avocados (1302), the avocados which had their coatings removed (1304), and the avocados which remained coated (1306). Two days after removal from cold storage, the untreated avocados (1302) exhibited a durometer stage reading of 2.88, the avocados which had their coatings removed (1304) exhibited a durometer stage reading of 2.97, and the avocados which remained coated (1306) exhibited a durometer stage reading of 2.37. Three days after removal from cold storage, the untreated avocados (1302) exhibited a durometer stage reading of 3.63, the avocados which had their coatings removed (1304) exhibited a durometer stage reading of 3.66, and the avocados which remained coated (1306) exhibited a durometer stage reading of 2.99. Four days after removal from cold storage, the untreated avocados (1302) exhibited a durometer stage reading of 4.00, the avocados which had their coatings removed (1304) exhibited a durometer stage reading of 4.13, and the avocados which remained coated (1306) exhibited a durometer stage reading of 3.53.

Various implementations of the compositions and methods have been described above. However, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modifications are in accordance with the variations of the disclosure. The implementations have been particularly shown and described, but it will be understood that various changes in form and details may be made. Accordingly, other implementations are within the scope of the following claims.

1. A composition comprising a first compound of Formula I-B and a second compound of Formula I-B, wherein Formula I-B is: 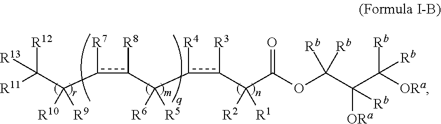
wherein:
each R^{a} is independently —H or —C₁-C₆ alkyl;
each R^{b} is independently selected from —H, —C₁-C₆ alkyl, or —OH;
R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;
R³, R⁴, R⁷, and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, —SR¹⁴, or halogen; or
R³ and R⁴ can combine with the carbon atoms to which they are attached to form a C₃-C₆ cycloalkyl, a C₄-C₆ cycloalkenyl, or a 3- to 6-membered ring heterocycle; and/or
R⁷ and R⁸ can combine with the carbon atoms to which they are attached to form a C₃-C₆ cycloalkyl, a C₄-C₆ cycloalkenyl, or a 3- to 6-membered ring heterocycle;
R¹⁴ and R¹⁵ are each independently, at each occurrence, —H, —C₁-C₆ alkyl, —C₂-C₆ alkenyl, or —C₂-C₆ alkynyl;
the symbol [symbol] represents a single bond or a cis or trans double bond;
n is 0, 1, 2, 3, 4, 5, 6, 7 or 8;
m is 0, 1, 2 or 3;
q is 0, 1, 2, 3, 4 or 5; and
r is 0, 1, 2, 3, 4, 5, 6, 7 or 8; wherein
a carbon chain length of the first compound of Formula I-B is different from a carbon chain length of the second compound of Formula I-B ; and
a combined mass of the first and second compounds of Formula I-B is at least about 50% of the total mass of the composition.

2. The composition of claim 1, wherein the first compound of Formula I-B comprises 2,3-dihydroxypropan-2-yl octadecanoate and the second compound of Formula I-B comprises 2,3-dihydroxypropan-2-yl palmitate.

3. The composition of claim 2, wherein the composition further comprises a salt.

4. The composition of claim 3, wherein the salt is an organic salt.

5. The composition of claim 1, wherein the first and second compounds of Formula I-B are each independently selected from the group consisting of:

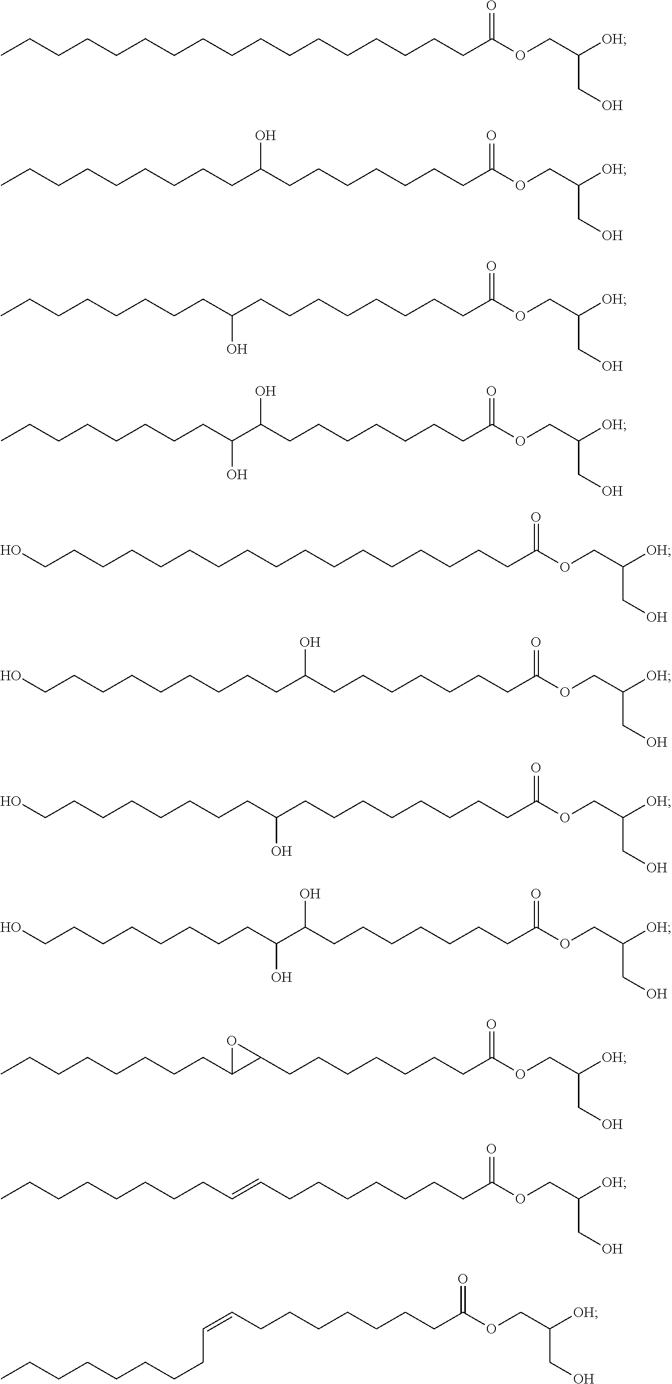

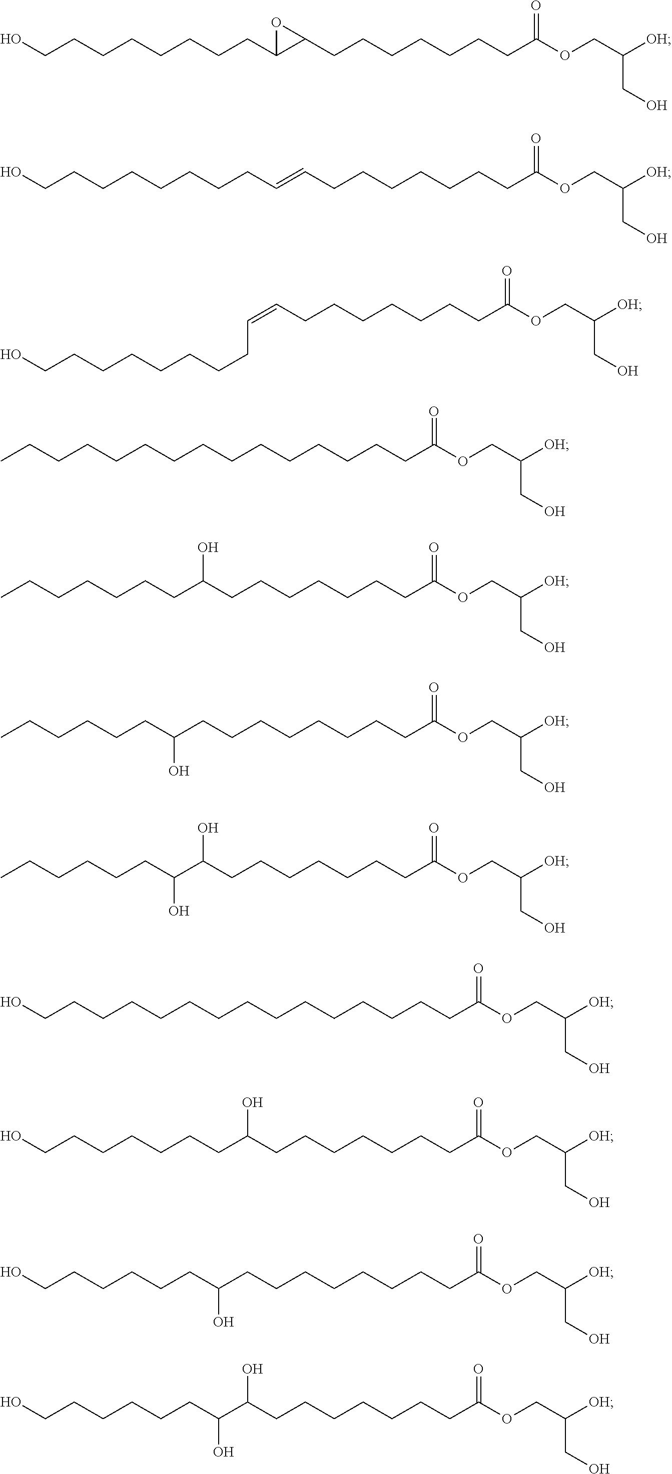

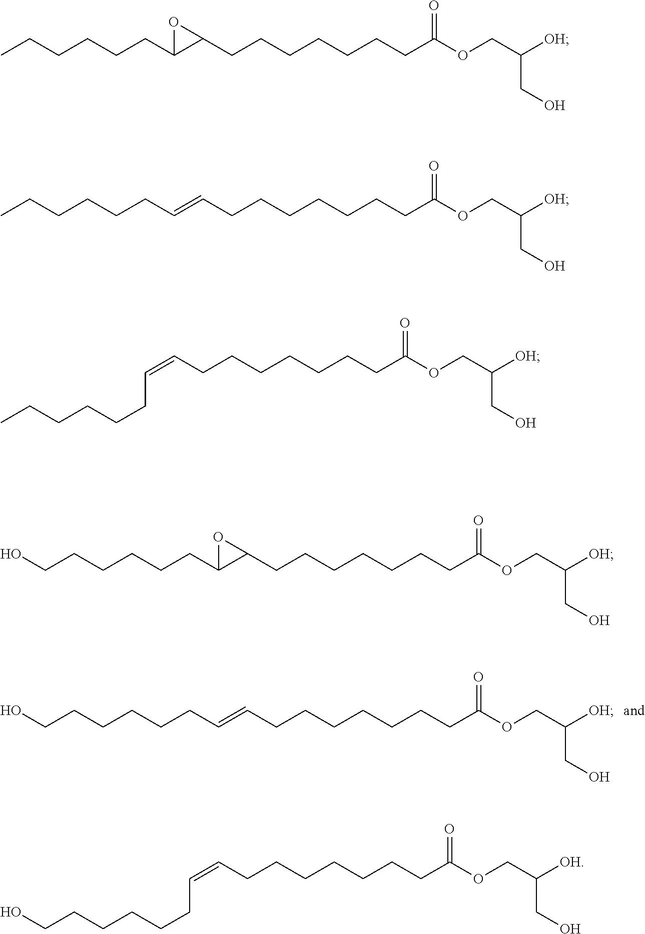 [SMILES: CCCCCCC1OC1CCCCCCCC(=O)OCC(O)CO ; CCCCCC/C=C/CCCCCCCC(=O)OCC(O)CO ; CCCCCC/C=C\CCCCCCCC(=O)OCC(O)CO ; O=C(CCCCCCCC1OC1CCCCCCO)OCC(O)CO ; O=C(CCCCCCC/C=C/CCCCCCO)OCC(O)CO ; O=C(CCCCCCC/C=C\CCCCCCO)OCC(O)CO]

6. The composition of claim 1, wherein the composition further comprises an organic salt.

7. The composition of claim 1, wherein the composition further comprises a fatty acid.

8. A composition comprising a first component and a second component different from the first component, the first component comprising a compound of Formula I-B, and the second component comprising a fatty acid or a second compound of Formula I-B, wherein Formula I-B is: 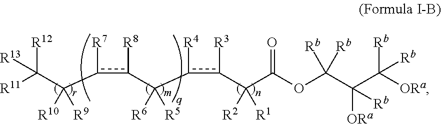
wherein:
each R^{a} is independently —H or —C₁-C₆ alkyl;
each R^{b} is independently selected from —H, —C₁-C₆ alkyl, or —OH;
R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;
R³, R⁴, R⁷, and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, —SR¹⁴, or halogen; or
R³ and R⁴ can combine with the carbon atoms to which they are attached to form a C₃-C₆ cycloalkyl, a C₄-C₆ cycloalkenyl, or a 3- to 6-membered ring heterocycle; and/or
R⁷ and R⁸ can combine with the carbon atoms to which they are attached to form a C₃-C₆ cycloalkyl, a C₄-C₆ cycloalkenyl, or a 3- to 6-membered ring heterocycle;
R¹⁴ and R¹⁵ are each independently, at each occurrence, —H, —C₁-C₆ alkyl, —C₂-C₆ alkenyl, or —C₂-C₆ alkynyl;
the symbol [symbol] represents a single bond or a cis or trans double bond;
n is 0,1,2,3,4,5,6,7 or 8;
m is 0,1,2 or 3;
q is 0, 1, 2, 3, 4 or 5; and
r is 0, 1, 2, 3, 4, 5, 6, 7 or 8; wherein
a combined mass of the first and second components is at least about 50% of the total mass of the composition.

9. The composition of claim 8, further comprising one or more additional components comprising a triglyceride, diglyceride, amide, amine, thiol, thioester, carboxylic acid, ether, aliphatic wax, alcohol, salt, acid, base, protein, or enzyme.

10. The composition of claim 9, wherein the additional component comprises an organic salt.

11. The composition of claim 10, wherein a carbon chain length of the first component is different from a carbon chain length of the second component.

12. The composition of claim 8, wherein the first component is selected from the group consisting of:

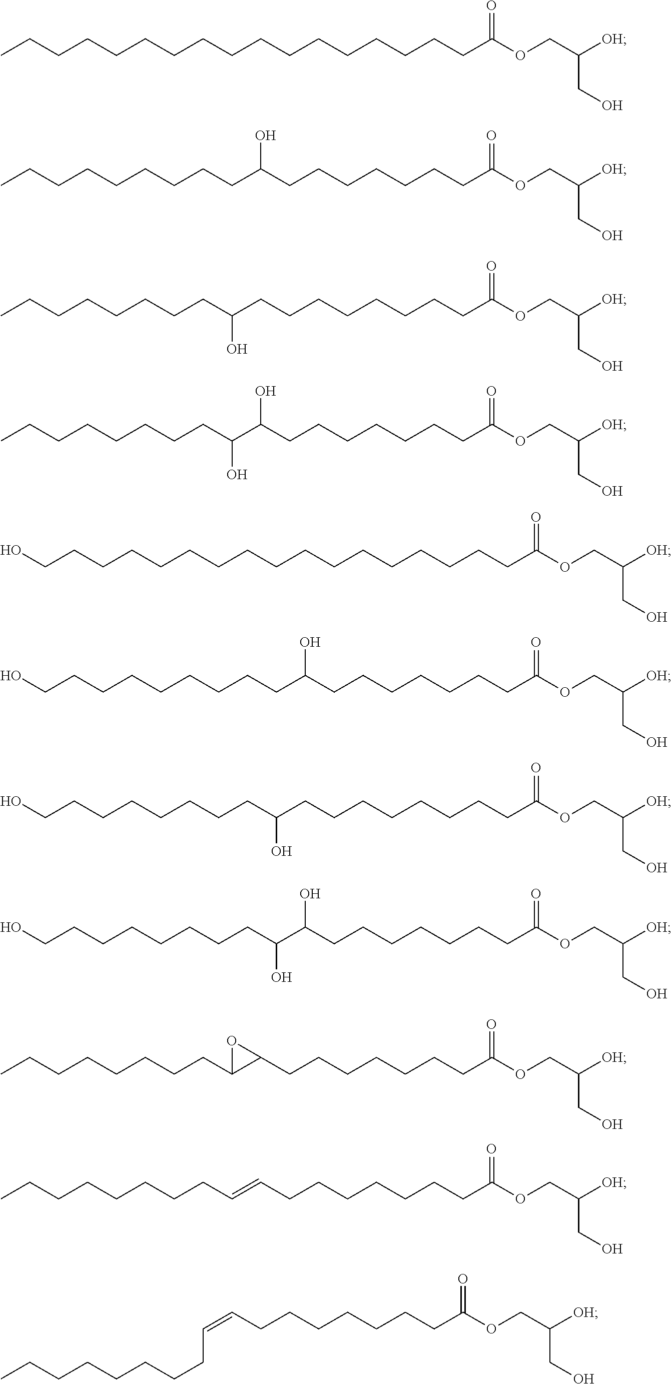

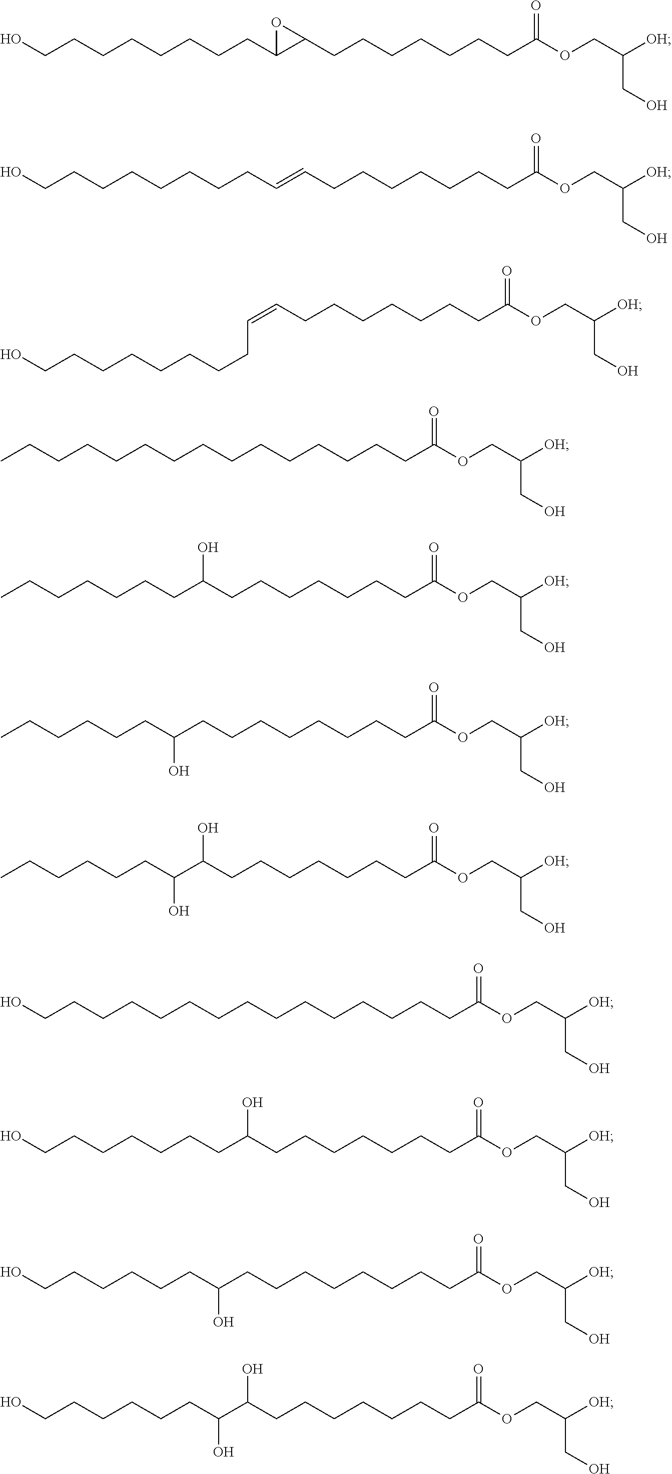

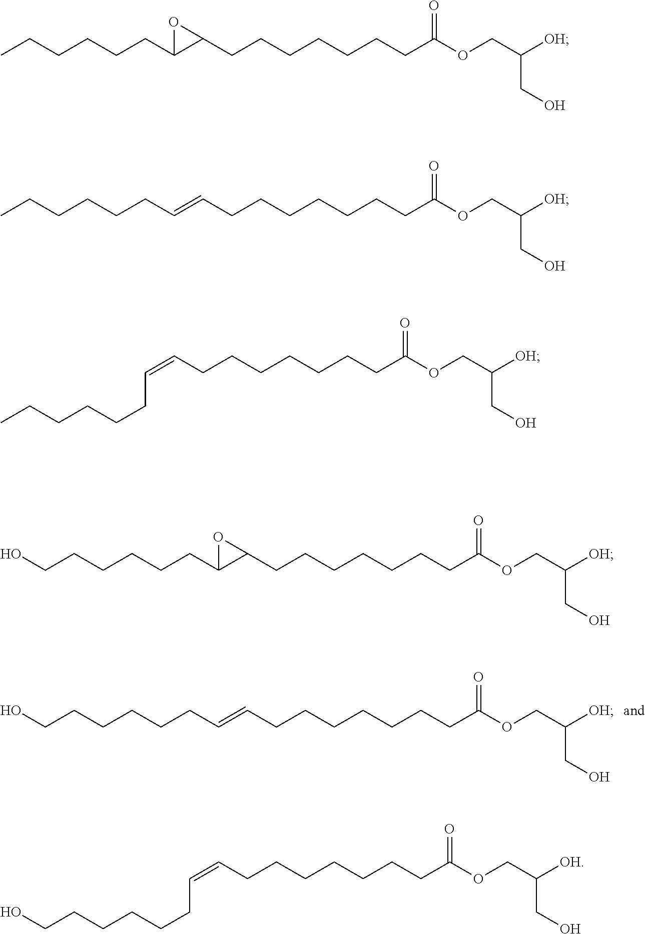 [SMILES: CCCCCCC1OC1CCCCCCCC(=O)OCC(O)CO ; CCCCCC/C=C/CCCCCCCC(=O)OCC(O)CO ; CCCCCC/C=C\CCCCCCCC(=O)OCC(O)CO ; O=C(CCCCCCCC1OC1CCCCCCO)OCC(O)CO ; O=C(CCCCCCC/C=C/CCCCCCO)OCC(O)CO ; O=C(CCCCCCC/C=C\CCCCCCO)OCC(O)CO]

13. The composition of claim 8, wherein the first component comprises 2,3-dihydroxypropan-2-yl octadecanoate and the second component comprises 2,3-dihydroxypropan-2-yl palmitate or palmitic acid.

14. The composition of claim 8, wherein a combined mass of the first and second components is at least 60% of the total mass of the composition.

15. A mixture comprising a composition in a solvent, the composition comprising a first component and a second component different from the first component, the first component comprising a compound of Formula I-B, and the second component comprising a fatty acid or a second compound of Formula I-B, wherein Formula I-B is: 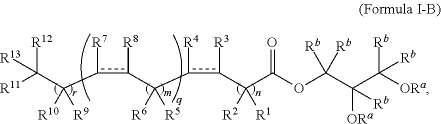
wherein:
each R^{a} is independently —H or —C₁-C₆ alkyl;
each R^{b} is independently selected from —H, —C₁-C₆ alkyl, or —OH;
R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;
R³, R⁴, R⁷, and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, —SR¹⁴, or halogen; or
R³ and R⁴ can combine with the carbon atoms to which they are attached to form a C₃-C₆ cycloalkyl, a C₄-C₆ cycloalkenyl, or a 3- to 6-membered ring heterocycle; and/or
R⁷ and R⁸ can combine with the carbon atoms to which they are attached to form a C₃-C₆ cycloalkyl, a C₄-C₆ cycloalkenyl, or a 3- to 6-membered ring heterocycle;
R¹⁴ and R¹⁵ are each independently, at each occurrence, —H, —C₁-C₆ alkyl, —C₂-C₆ alkenyl, or —C₂-C₆ alkynyl;
the symbol [symbol] represents a single bond or a cis or trans double bond;
n is 0, 1, 2, 3, 4, 5, 6, 7 or 8;
m is 0, 1, 2 or 3;
q is 0, 1, 2, 3, 4 or 5; and
r is 0, 1, 2, 3, 4, 5, 6, 7 or 8; wherein
a combined mass of the first and second components is at least about 50% of the total mass of the composition.

16. The mixture of claim 15, wherein the solvent comprises ethanol, methanol, acetone, isopropanol, ethyl acetate, water, or combinations thereof.

17. The mixture of claim 15, wherein the solvent comprises ethanol.

18. The mixture of claim 15, wherein the solvent comprises water.

19. The mixture of claim 18, wherein the mixture further comprises an organic salt.

20. The mixture of claim 19, wherein the first component comprises 2,3-dihydroxypropan-2-yl octadecanoate and the second component comprises 2,3-dihydroxypropan-2-yl palmitate.

21. The mixture of claim 15, wherein a concentration of the composition in the solvent is in a range of 0.1 to 200 mg/mL.

22. The mixture of claim 15, wherein the first component comprises 2,3-dihydroxypropan-2-yl octadecanoate and the second component comprises 2,3-dihydroxypropan-2-yl palmitate or palmitic acid.

23. The mixture of claim 15, wherein a carbon chain length of the first component is different from a carbon chain length of the second component.

24. A method of forming a coating on a product, the method comprising:
(i) applying a mixture to a surface of the product, the mixture comprising a composition in a solvent, the composition comprising a first component and a second component different from the first component, the first component comprising a compound of Formula I-B, and the second component comprising a fatty acid or a second compound of Formula I-B, wherein Formula I-B is: 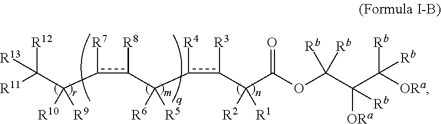
wherein:
each R^{a} is independently —H or —C₁-C₆ alkyl;
each R^{b} is independently selected from —H, —C₁-C₆ alkyl, or —OH;
R¹, R², R⁵, R⁶, R⁹, R¹⁰, R¹¹, R¹² and R¹³ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with —OR¹⁴, —NR¹⁴R¹⁵, —SR¹⁴, or halogen;
R³, R⁴, R⁷, and R⁸ are each independently, at each occurrence, —H, —OR¹⁴, —NR¹⁴R¹⁵, —SR¹⁴, halogen, —C₁-C₆ alkyl, —C₂-C₆ alkenyl, —C₂-C₆ alkynyl, —C₃-C₇ cycloalkyl, aryl, or heteroaryl, wherein each alkyl, alkenyl, alkynyl, cycloalkyl, aryl, or heteroaryl is optionally substituted with one or more —OR¹⁴, —NR¹⁴R¹⁵, —SR¹⁴, or halogen; or
R³ and R⁴ can combine with the carbon atoms to which they are attached to form a C₃-C₆ cycloalkyl, a C₄-C₆ cycloalkenyl, or a 3- to 6-membered ring heterocycle; and/or
R⁷ and R⁸ can combine with the carbon atoms to which they are attached to form a C₃-C₆ cycloalkyl, a C₄-C₆ cycloalkenyl, or a 3- to 6-membered ring heterocycle;
R¹⁴ and R¹⁵ are each independently, at each occurrence, —H, —C₁-C₆ alkyl, —C₂-C₆ alkenyl, or —C₂-C₆ alkynyl;
the symbol [symbol] represents a single bond or a cis or trans double bond;
n is 0, 1, 2, 3, 4, 5, 6, 7 or 8;
m is 0, 1, 2 or 3;
q is 0, 1, 2, 3, 4 or 5; and
r is 0, 1, 2, 3, 4, 5, 6, 7 or 8; wherein
a combined mass of the first and second components is at least about 50% of the total mass of the coating agent; and
(ii) causing the composition to solidify on the surface to form the coating.

25. The method of claim 24, wherein the product is an agricultural product.

26. The method of claim 24, wherein the product is a fruit or vegetable.

27. The method of claim 24, wherein the product is an avocado.

28. The method of claim 24, wherein causing the composition to solidify on the surface comprises at least partially removing the solvent from the surface.

29. The method of claim 28, wherein the at least partially removing the solvent from the surface comprises allowing the solvent to evaporate.

30. The method of claim 24, wherein the coating has an average thickness in the range of 0.1 microns to 10 microns.
